SEQUENCE LISTING (98 sequences)

SEQ ID NO: 1 - RNA, 167 nt, Mus musculus
  AS Uchl1 SINEB2
cagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcucc

SEQ ID NO: 2 - RNA, 77 nt, Mus musculus
  nt 44-120 of inverted SINE B2 transposable element derived from AS Uchl1
gaacuggaguuauacgguaaccucguggugguugugaaccaccauguggauggauauugaguuccaaacacuggucc

SEQ ID NO: 3 - RNA, 29 nt, Mus musculus
  nt 64-92 of inverted SINE B2 transposable element derived from AS Uchl1
ccucguggugguugugaaccaccaugugg

SEQ ID NO: 4 - RNA, 61 nt, Mus musculus
  nt 52-112 of inverted SINE B2 transposable element derived from AS Uchl1
guuauacgguaaccucguggugguugugaaccaccauguggauggauauugaguuccaaac

SEQ ID NO: 5 - RNA, 98 nt, Mus musculus
  nt 36-133 of inverted SINE B2 transposable element derived from AS Uchl1
aucccccagaacuggaguuauacgguaaccucguggugguugugaaccaccauguggauggauauugaguuccaaacacugguccugugcaagagcau

SEQ ID NO: 6 - RNA, 129 nt, Mus musculus
  nt 22-150 of inverted SINE B2 transposable element derived from AS Uchl1
gaagagggcauuggaucccccagaacuggaguuauacgguaaccucguggugguugugaaccaccauguggauggauauugaguuccaaacacugguccugugcaagagcauccagugcucuuaagugc

SEQ ID NO: 7 - RNA, 183 nt, Mus musculus
  nt 1-183 of inverted SINE B2 transposable element derived from AS Uchl1
gggcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaagcu

SEQ ID NO: 8 - RNA, 67 nt, Mus musculus
  nt 44-110 of inverted SINE B2 transposable element derived from AS Uchl1
gaacuggaguuauacgguaaccucguggugguugugaaccaccauguggauggauauugaguuccaa

SEQ ID NO: 9 - RNA, 107 nt, Mus musculus
  nt 34-140 of inverted SINE B2 transposable element derived from AS Uchl1
ggaucccccagaacuggaguuauacgguaaccucguggugguugugaaccaccauguggauggauauugaguuccaaacacugguccugugcaagagcauccagugc

SEQ ID NO: 10 - RNA, 127 nt, Mus musculus
  nt 24-150 of inverted SINE B2 transposable element derived from AS Uchl1
agagggcauuggaucccccagaacuggaguuauacgguaaccucguggugguugugaaccaccauguggauggauauugaguuccaaacacugguccugugcaagagcauccagugcucuuaagugc

SEQ ID NO: 11 - RNA, 38 nt, Mus musculus
  nt 59-96 of inverted SINE B2 transposable element derived from AS Uchl1
gguaaccucguggugguugugaaccaccauguggaugg

SEQ ID NO: 12 - RNA, 180 nt, Artificial Sequence
  G60A mutant of inverted SINE B2 transposable element derived from AS Uchl1
cagugcuagaggaggucagaagagggcauuggaucccccagaacuggaguuauacgauaaccucguggugguugugaaccaccauguggauggauauugaguuccaaacacugguccugugcaagagcauccagugcucuuaagugcugagccaucucuuuagcuccagucucuuaagcu

SEQ ID NO: 13 - RNA, 180 nt, Artificial Sequence
  G60C mutant of inverted SINE B2 transposable element derived from AS Uchl1
cagugcuagaggaggucagaagagggcauuggaucccccagaacuggaguuauacgcuaaccucguggugguugugaaccaccauguggauggauauugaguuccaaacacugguccugugcaagagcauccagugcucuuaagugcugagccaucucuuuagcuccagucucuuaagcu

SEQ ID NO: 14 - RNA, 180 nt, Artificial Sequence
  A51C mutant of inverted SINE B2 transposable element derived from AS Uchl1
cagugcuagaggaggucagaagagggcauuggaucccccagaacuggcguuauacgguaaccucguggugguugugaaccaccauguggauggauauugaguuccaaacacugguccugugcaagagcauccagugcucuuaagugcugagccaucucuuuagcuccagucucuuaagcu

SEQ ID NO: 15 - RNA, 180 nt, Artificial Sequence
  C47G and G117C mutant of inverted SINE B2 transposable element derived from AS Uchl1
cagugcuagaggaggucagaagagggcauuggaucccccagaaguggaguuauacgguaaccucguggugguugugaaccaccauguggauggauauugaguuccaaacacugcuccugugcaagagcauccagugcucuuaagugcugagccaucucuuuagcuccagucucuuaagcu

SEQ ID NO: 16 - RNA, 180 nt, Artificial Sequence
  46-49/115-118 stem swap mutant of inverted SINE B2 transposable element derived from AS Uchl1
cagugcuagaggaggucagaagagggcauuggaucccccagauggugaguuauacgguaaccucguggugguugugaaccaccauguggauggauauugaguuccaaacacgucaccugugcaagagcauccagugcucuuaagugcugagccaucucuuuagcuccagucucuuaagcu

SEQ ID NO: 17 - RNA, 180 nt, Artificial Sequence
  50-53 and 112-114 base swap mutant of inverted SINE B2 transposable element derived from AS Uchl1
cagugcuagaggaggucagaagagggcauuggaucccccagaacugcacuauacgguaaccucguggugguugugaaccaccauguggauggauauugaguuccaaaugagugguccugugcaagagcauccagugcucuuaagugcugagccaucucuuuagcuccagucucuuaagcu

SEQ ID NO: 18 - RNA, 116 nt, Artificial Sequence
  nt 27-142 of inverted SINE B2 transposable element derived from AS Uchl1
gggcauuggaucccccagaacuggaguuauacgguaaccucguggugguugugaaccaccauguggauggauauugaguuccaaacacugguccugugcaagagcauccagugcuc

SEQ ID NO: 19 - RNA, 77 nt, Artificial Sequence
  nt 44-120 A45G mutant of inverted SINE B2 transposable element derived from AS Uchl1
ggacuggaguuauacgguaaccucguggugguugugaaccaccauguggauggauauugaguuccaaacacuggucc

SEQ ID NO: 20 - RNA, 77 nt, Artificial Sequence
  nt 44-120 A51C mutant of inverted sine B2 transposable element derived from AS Uchl1
gaacuggcguuauacgguaaccucguggugguugugaaccaccauguggauggauauugaguuccaaacacuggucc

SEQ ID NO: 21 - RNA, 107 nt, Mus musculus
  AS Txnip SINEB2
gaugccuuagaaguggaguuaagaguugugagcugccguuuuuugguucugggacucgaacucguuuccucugauacuaucaaccaccaagccaucucuucagcccc

SEQ ID NO: 22 - RNA, 131 nt, Mus musculus
  AS Uxt SINEB2 a
gccagaagaaguugugggauucccuggaacuggagcaaccaacaguuugugugcaccauguggguaaugggaaucgaaccuggguccucuauaagacuggccagugcucuuaacuacugaggugcauuucu

SEQ ID NO: 23 - RNA, 187 nt, Mus musculus
  AS Uxt SINEB2 b
uuauuuuaaauauaugaguauuucaccugcauaggcgcacaguacccacagagacuagaagaggguggcagaucuccugagacuggaguuaaugcuugugagcugccauguggaugcuggaaaucaaacccagguccuuuggaaggcaggcaggugcucuuaaucauggaagcaucucuucagcucc

SEQ ID NO: 24 - RNA, 131 nt, Mus musculus
  AS Gadd45alpha SINEB2 a
cagcgacaucagaagaggauauuggaucccauuacagaugguugaaggccaccaugucguugcugggaaugaacucaagaccucuggaagagcagucagugcucuuaaccucugagccaucucuccagccc

SEQ ID NO: 25 - RNA, 114 nt, Mus musculus
  AS Gadd45alpha SINEB2 b
auccccuccaaagcucaagaugguuguaagccacccugugauugcugggauuugaacucaagaccuccggaagagcaauuagugcucuuaaccgcugagcaaucucuccagccc

SEQ ID NO: 26 - RNA, 357 nt, Mus musculus
  AS Uchl1 SINEB2 + AS Uxt SINEB2 b
gugcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccuuauuuuaaauauaugaguauuucaccugcauaggcgcacaguacccacagagacuagaagaggguggcagaucuccugagacuggaguuaaugcuugugagcugccauguggaugcuggaaaucaaacccagguccuuuggaaggcaggcaggugcucuuaaucauggaagcaucucuucagcucc

SEQ ID NO: 27 - RNA, 532 nt, Mus musculus
  3x AS Uchl1 SINEB2
gugcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccgugcgaauucggugcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccgugcgaauucggugcagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcucc

SEQ ID NO: 28 - RNA, 228 nt, Mus musculus
  SINEB2/B3 consensus sequence from RepBase
uuuuuuuaaaaauuuauuuuuauuuuauguguaugaguguuuugccugcauguaugucuguguaccacgugcgugccuggugcccgcggaggccagaagagggcgucggauccccuggaacuggaguuacagaugguugugagccgccaugugggugcugggaaucgaacccggguccucuggaagagcagccagugcucuuaaccgcugagccaucucuccagcccc

SEQ ID NO: 29 - RNA, 214 nt, Mus musculus
  AS Nars2 SINEB2/B3
uuuuuuuuacuuguauagguguuuugccugcauguguaucuaucuauguaccgaauauguuccugguauccacagagaccaaaaguggauguuguaucuccugaaauuggagucauagacaguuaugagcugccauuugagugcuuggaauagaacccagguccucuuaaagagcauccagugcucuuaaaaacugagacaucucuguagccuc

SEQ ID NO: 30 - RNA, 200 nt, Mus musculus
  AS Abhd11 SINEB2/B3
uuuauuuugcuuuaugugucugaguguuugcuugaauguaugucuguguaccacgccuguaccuugugccuucagaguugagaggagggcauaggaucuccuggaacuggaauugcaggugguugugagccacccuguggguccuggggaccauacuccagcaagaacaucaugugcucuuaauuccugagucuccaacc

SEQ ID NO: 31 - RNA, 214 nt, Mus musculus
  AS Ebp4.9 SINEB2/B3
uuuauuuacuuaucuuuauguguaugaguguguugucagacuguuaugucugugugucacaugcaugccugcuguucauggaguccagaagagggcaucggauccccuggaacuggaguuacagaugaguggccaugugaauguuaagaaccaaaccuggguccucugaaagagcagacaaugcucuuaacuacugagcugucucuccagcccc

SEQ ID NO: 32 - RNA, 205 nt, Mus musculus
  AS Wfdc5 SINEB2/B3
uuauuuuauucguguaaguguuuugccagcaucuaugucuucgcacuaugugcaggucuggugccugagggguccagacgagagcacugggucuccgggaacuggaguuacagaucauugugagccaccaugugggugcagggaaucgaaccugggaccucuggaggagcagccacugcucuuaaccacuacacuauuucuccag

SEQ ID NO: 33 - RNA, 121 nt, Mus musculus
  AS Pgbd1 SINEB2/B3
ucuguggaccacuguguacagaagccugagaaggcuagcagauccccagaacuggaacugugagacgcugugcuauggaggugcuaggaacugaaaauggauggguccucugcaagagcag

SEQ ID NO: 34 - RNA, 191 nt, Mus musculus
  AS Gsk3b SINEB2/B3
uuguuuuaauugaauggcuauaggguguuucuucuguauguauaucuauguuugguaccuacagaggcaucagauccucuggaacuguaguugcugacaguugugagcugucauggggaugcuggaauugaaccuggauccuaugaaagaacagccaguguucuuaaccgcugagcuaucucuccaggccc

SEQ ID NO: 35 - RNA, 205 nt, Mus musculus
  AS Rhod SINEB2_Mm2
uuuuuuuuuuaauuuuaaaaaaaaagauuuuauuuauuuauuuuauauaugaugaguacacugucacucuuuucagacacccuagaaaaggggggcaucagaucccauuacagaugguugugagccacaugguugcugggaauugaccucaggaccucugaaagagcagucagugcucucaaccuuugagucaucucuccagccc

SEQ ID NO: 36 - RNA, 190 nt, Mus musculus
  AS Rhod SINEB2/B3A
auguauaucuguaaugggacauacucacauacaugggcacgugaguauaaaaggccagaagagagcacuggacccucuggaguugagauucuaagcaguugugaaccaucugauguaggugcugggaacugaacuuggguccuuugcuagagaaguaugucucuuaaccacugagccguaucuccauccc

SEQ ID NO: 37 - RNA, 169 nt, Mus musculus
  AS E4f1 SINEB2_Mm2
uaaagauuuauucauuaaguacacuguagcuaucuucagacgcaucagaagagggcgucagaucucuuuacaggugguugugagccaccaugugguugcuggaauuugaacucaggaccuucaaaagagcagucaguguucuuaaccgcugagccaucucuccaacccc

SEQ ID NO: 38 - RNA, 159 nt, Mus musculus
  AS E4f1 SINEB2_Mm1t
uuauuuauuauaaguacacuguagcugucuucagacacaacaaaagagggcgucagaucucauuacaggugguugagccaccaugugguugcugggauuugaacucaggaccuucagaacagucagugcucuuacccacugagccagcgagccagcccc

SEQ ID NO: 39 - RNA, 77 nt, Artificial Sequence
  44-120 U78G SINEUP
gaacuggaguuauacgguaaccucguggugguugggaaccaccauguggauggauauugaguuccaaacacuggucc

SEQ ID NO: 40 - RNA, 77 nt, Artificial Sequence
  44-120 GUG77-79CCC SINEUP
gaacuggaguuauacgguaaccucguggugguucccaaccaccauguggauggauauugaguuccaaacacuggucc

SEQ ID NO: 41 - RNA, 77 nt, Artificial Sequence
  44-120 SINEUP strong
ggaccggaguuauacgguaaccgcguggugguugugaaccaccacgcggauggauauugaguuccaaacaccggucc

SEQ ID NO: 42 - RNA, 77 nt, Artificial Sequence
  44-120 SINEUP weak
gaacuagaguuauacgguaaccacauggugguugugaaccaccauguggauggauauugaguuccaaacacuaguuc

SEQ ID NO: 43 - RNA, 206 nt, Artificial Sequence
  SINEUP 071 and miniSINEUP 071 effector domain
uuauuuuaaauauaugaguauuucaccugcauaggcgcacaguacccacagagacuagaagaggguaguagauccccuagaacuggaguuauacgguaaccucguggugguugugagcuaccauguggauggauacugggaaucaaacccagguccuguggaaggcaggcaggugcucucaagcacugagccaucucuucagcucc

SEQ ID NO: 44 - RNA, 206 nt, Artificial Sequence
  SINEUP 072 and miniSINEUP 072 effector domain
uuauuuuaaauauaugaguauuucaccugcauaggcgcacagugcucaaggagaucagaagagggcaucagaucuccugagacuggaguuauacgguaaccucgugaugguugugaacuaccauguggauggauauugaguuccaaacacagguccugugcaagagcagcaggugcucuuaagcacggaaccaucucuuuagcucc

SEQ ID NO: 45 - RNA, 156 nt, Artificial Sequence
  SINEUP 073 and miniSINEUP 073 effector domain
gaggcuagaagaggguaucagauccccugagacuggaguuauacgguaaccucguggugguugugagccaccauguggauggauacugagaaccaaacccugguccugugcaagagcaucaggugcucuuaagcacggaaccaucucuucagcucc

SEQ ID NO: 46 - RNA, 68 nt, Artificial Sequence
  SINEUP 074 and miniSINEUP 074 effector domain
guccugugcaagagcaucgaacucggugcucuuaagcacagaagccaccaagccaucucuucagcccc

SEQ ID NO: 47 - RNA, 110 nt, Artificial Sequence
  SINEUP 075 and miniSINEUP 075 effector domain
cagugcuagaggaggucagaagagggcaucccccagccucguggugguugugaaccaccauguggcugugcaagagcaugcucuuaagugcugagccaucucuuuagcuc

SEQ ID NO: 48 - RNA, 126 nt, Artificial Sequence
  24-150 effector domain
gagggcauuggaucccccagaacuggaguuauacgguaaccucguggugguugugaaccaccauguggauggauauugaguuccaaacacugguccugugcaagagcauccagugcucuuaagugc

SEQ ID NO: 49 - RNA, 89 nt, Artificial Sequence
  C_34-122 effector domain
ggaucccccagaacuggaguuauacgguaaccucguggugguugugaaccaccauguggauggauauugaguuccaaacacugguccug

SEQ ID NO: 50 - RNA, 72 nt, Artificial Sequence
  TM effector domain
ugcuagaggaggucagaagagggcauuggaugcaaauccagugcucuuaagugcugagccaucucuuuagcu

SEQ ID NO: 51 - RNA, 94 nt, Artificial Sequence
  MC2 effector domain
gagggcauuggaucccccagaacuggaguuauacgguaacgauggauauugaguuccaaacacugguccugugcaagagcauccagugcucuua

SEQ ID NO: 52 - RNA, 13 nt, Artificial Sequence
  linker
aucugcagaauuc

SEQ ID NO: 53 - RNA, 383 nt, Hepatitis C virus
  HCV(d) IRES
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguugggucgcgaaaggccuugugguacugccugauagggugcuugcgagugccccgggaggucucguagaccgugcaccaugagcacgaauccuaaaccucaaagaaaaaccaaacguaac

SEQ ID NO: 54 - RNA, 383 nt, Hepatitis C virus
  HCV(i) IRES
guuacguuugguuuuucuuugagguuuaggauucgugcucauggugcacggucuacgagaccucccggggcacucgcaagcacccuaucaggcaguaccacaaggccuuucgcgacccaacacuacucggcuagcagucuugcgggggcacgcccaaaucuccaggcauugagcggguuuauccaagaaaggacccggucguccuggcaauuccgguguacucaccgguuccgcagaccacuauggcucucccgggaggggggguccuggaggcugcacgacacucauacuaacgccauggcuagacgcuuucugcgugaagacaguaguuccucacaggggagugauucaugguggagugucgcccccaucagggggcuggc

SEQ ID NO: 55 - RNA, 312 nt, Human poliovirus
  Polio(d) IRES
augagucuggacaucccucaccggugacggugguccaggcugcguuggcggccuaccuauggcuaacgccaugggacgcuaguugugaacaaggugugaagagccuauugagcuacauaagaauccuccggccccugaaugcggcuaaucccaaccucggagcagguggucacaaaccagugauuggccugucguaacgcgcaaguccguggcggaaccgacuacuuuggguguccguguuuccuuuuauuuuauuguggcugcuuauggugacaaucacagauuguuaucauaaagcgaauuggauuggcc

SEQ ID NO: 56 - RNA, 312 nt, Human poliovirus
  Polio(i) IRES
ggccaauccaauucgcuuuaugauaacaaucugugauugucaccauaagcagccacaauaaaauaaaaggaaacacggacacccaaaguagucgguuccgccacggacuugcgcguuacgacaggccaaucacugguuugugaccaccugcuccgagguugggauuagccgcauucaggggccggaggauucuuauguagcucaauaggcucuucacaccuuguucacaacuagcgucccauggcguuagccauagguaggccgccaacgcagccuggaccaccgucaccggugagggauguccagacucau

SEQ ID NO: 57 - RNA, 576 nt, Encephalomyocarditis virus
  EMCV(d) IRES
cccccccucucccuccccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaa

SEQ ID NO: 58 - RNA, 576 nt, Encephalomyocarditis virus
  ECMV(i) IRES
uuaucaucguguuuuucaaaggaaaaccacguccccgugguucggggggccuagacguuuuuuaaccucgacuaaacacauguaaagcaugugcaccgaggccccagaucagaucccauacaaugggguaccuucugggcauccuucagccccuuguugaauacgcuugaggagagccauuugacucuuuccacaacuauccaacucacaacguggcacugggguugugccgccuuugcagguguaucuuauacacguggcuuuuggccgcagaggcaccugucgccaggugggggguuccgcugccugcaaagggucgcuacagacguuguuugucuucaagaagcuuccagaggaacugcuuccuucacgacauucaacagaccuugcauuccuuuggcgagaggggaaagaccccuaggaaugcucgucaagaagacagggccagguuuccgggcccucacauugccaaaagacggcaauaugguggaaaauaacauauagacaaacgcacaccggccuuauuccaagcggcuucggccaguaacguuaggggggggggagggagaggggggg

SEQ ID NO: 59 - RNA, 192 nt, Cricket paralysis virus
  CrPV(d) IRES
aaagcaaaaaugugaucuugcuuguaaauacaauuuugagagguuaauaaauuacaaguagugcuauuuuuguauuuagguuagcuauuuagcuuuacguuccaggaugccuaguggcagccccacaauauccaggaagcccucucugcgguuuuucagauuagguagucgaaaaaccuaagaaauuuaccu

SEQ ID NO: 60 - RNA, 192 nt, Cricket paralysis virus
  CrPV(i) IRES
agguaaauuucuuagguuuuucgacuaccuaaucugaaaaaccgcagagagggcuuccuggauauuguggggcugccacuaggcauccuggaacguaaagcuaaauagcuaaccuaaauacaaaaauagcacuacuuguaauuuauuaaccucucaaaauuguauuuacaagcaagaucacauuuuugcuuu

SEQ ID NO: 61 - RNA, 231 nt, Homo sapiens
  Apaf-1(d) IRES
cagagauccaggggaggcgccugugaggcccggaccugccccggggcgaaggguauguggcgagacagagcccugcaccccuaauucccgguggaaaacuccuguugccguuucccuccaccggccuggagucucccagucuugucccggcagugccgcccuccccacuaagaccuaggcgcaaaggcuuggcucaugguugacagcucagagagagaaagaucugaggga

SEQ ID NO: 62 - RNA, 231 nt, Homo sapiens
  Apaf-1(i) IRES
ucccucagaucuuucucucucugagcugucaaccaugagccaagccuuugcgccuaggucuuaguggggagggcggcacugccgggacaagacugggagacuccaggccgguggagggaaacggcaacaggaguuuuccaccgggaauuaggggugcagggcucugucucgccacauacccuucgccccggggcagguccgggccucacaggcgccuccccuggaucucug

SEQ ID NO: 63 - RNA, 460 nt, Homo sapiens
  ELG-1(d) IRES
acuuuuggugggcauuuaaaaauguguguguauguguauauauguauguguauguauguguauauauguauauguauguauguaucgcguguauguguguauguaugcauguguauguauguauaugcauguauguguauguguauauauguauguguguguauguauauguguguguauguguauguguguguguauguguguguguauguauguauguauguauauguauuauacacauauacacauauugguuuuuuuaaucauuugagaguuaguugaagauaaaaacccaucaccccuaaauguauuccaaagaauaagaacauuguuuuauacauagcacacuuaacaaaaucaagaaauuuaacauuaauacaguacuguuaccuaauccguagucgauuuucaaauuuugucaguuguuccaauaauguccuuuauauauuccccgcccagc

SEQ ID NO: 64 - RNA, 460 nt, Homo sapiens
  ELG-1(i) IRES
gcugggcggggaauauauaaaggacauuauuggaacaacugacaaaauuugaaaaucgacuacggauuagguaacaguacuguauuaauguuaaauuucuugauuuuguuaagugugcuauguauaaaacaauguucuuauucuuuggaauacauuuaggggugauggguuuuuaucuucaacuaacucucaaaugauuaaaaaaaccaauauguguauauguguauaauacauauacauacauacauacauacacacacacauacacacacacauacacauacacacacauauacauacacacacauacauauauacacauacacauacaugcauauacauacauacacaugcauacauacacacauacacgcgauacauacauacauauacauauauacacauacauacacauacauauauacacauacacacacauuuuuaaaugcccaccaaaagu

SEQ ID NO: 65 - RNA, 395 nt, Homo sapiens
  cMYC full length(d) IRES
aauuccagcgagaggcagagggagcgagcgggcggccggcuaggguggaagagccgggcgagcagagcugcgcugcgggcguccugggaagggagauccggagcgaauagggggcuucgccucuggcccagcccucccgcuugaucccccaggccagcgguccgcaacccuugccgcauccacgaaacuuugcccauagcagcgggcgggcacuuugcacuggaacuuacaacacccgagcaaggacgcgacucucccgacgcggggaggcuauucugcccauuuggggacacuuccccgccgcugccaggacccgcuucucugaaaggcucuccuugcagcugcuuagacgcuggauuuuuuucggguaguggaaaaccagcagccucccgcga

SEQ ID NO: 66 - RNA, 395 nt, Homo sapiens
  cMYC full length(i) IRES
ucgcgggaggcugcugguuuuccacuacccgaaaaaaauccagcgucuaagcagcugcaaggagagccuuucagagaagcggguccuggcagcggcggggaaguguccccaaaugggcagaauagccuccccgcgucgggagagucgcguccuugcucggguguuguaaguuccagugcaaagugcccgcccgcugcuaugggcaaaguuucguggaugcggcaaggguugcggaccgcuggccugggggaucaagcgggagggcugggccagaggcgaagcccccuauucgcuccggaucucccuucccaggacgcccgcagcgcagcucugcucgcccggcucuuccacccuagccggccgcccgcucgcucccucugccucucgcuggaauu

SEQ ID NO: 67 - RNA, 48 nt, Homo sapiens
  cMYC short variant(d) IRES
gggcacuuugcacuggaacuuacaacacccgagcaaggacgcgacucu

SEQ ID NO: 68 - RNA, 48 nt, Homo sapiens
  cMYC short variant(i) IRES
agagucgcguccuugcucggguguuguaaguuccagugcaaagugccc

SEQ ID NO: 69 - RNA, 71 nt, Homo sapiens
  DMD(d) IRES
guacugacaucguagauggaaaucauaaacugacucuugguuugauuuggaauauaauccuccacuggcag

SEQ ID NO: 70 - RNA, 71 nt, Homo sapiens
  DMD(i) IRES
cugccaguggaggauuauauuccaaaucaaaccaagagucaguuuaugauuuccaucuacgaugucaguac

SEQ ID NO: 71 - RNA, 303 nt, Hepatitis C virus
  HCV delta II(d) IRES
gccagcccccugaugggggcgacacuccaccaugaaucacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguugggucgcgaaaggccuugugguacugccugauagggugcuugcgagugccccgggaggucucguagaccgugcaccaugagcacgaauccuaaaccucaaagaaaaaccaaacguaac

SEQ ID NO: 72 - RNA, 367 nt, Hepatitis C virus
  HCV delta IIIa(d) IRES
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaaauugccaggacgaccggguccuuucuuggauaaacccgcucaaugccuggagauuugggcgugcccccgcaagacugcuagccgaguaguguugggucgcgaaaggccuugugguacugccugauagggugcuugcgagugccccgggaggucucguagaccgugcaccaugagcacgaauccuaaaccucaaagaaaaaccaaacguaac

SEQ ID NO: 73 - RNA, 356 nt, Hepatitis C virus
  HCV delta IIId(d) IRES
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuacuugugguacugccugauagggugcuugcgagugccccgggaggucucguagaccgugcaccaugagcacgaauccuaaaccucaaagaaaaaccaaacguaac

SEQ ID NO: 74 - RNA, 330 nt, Hepatitis C virus
  HCV delta IV(d) IRES
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguugggucgcgaaaggccuugugguacugccugauagggugcuugcgagugccccgggaggucucgua

SEQ ID NO: 75 - RNA, 383 nt, Hepatitis C virus
  HCV G266->C(d) IRES
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguucggucgcgaaaggccuugugguacugccugauagggugcuugcgagugccccgggaggucucguagaccgugcaccaugagcacgaauccuaaaccucaaagaaaaaccaaacguaac

SEQ ID NO: 76 - RNA, 383 nt, Hepatitis C virus
  HCV U228->C(d) IRES
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauucgggcgugcccccgcaagacugcuagccgaguaguguugggucgcgaaaggccuugugguacugccugauagggugcuugcgagugccccgggaggucucguagaccgugcaccaugagcacgaauccuaaaccucaaagaaaaaccaaacguaac

SEQ ID NO: 77 - RNA, 383 nt, Hepatitis C virus
  HCV G267->C(d) IRES
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguugcgucgcgaaaggccuugugguacugccugauagggugcuugcgagugccccgggaggucucguagaccgugcaccaugagcacgaauccuaaaccucaaagaaaaaccaaacguaac

SEQ ID NO: 78 - RNA, 383 nt, Hepatitis C virus
  HCV G268->C(d) IRES
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguuggcucgcgaaaggccuugugguacugccugauagggugcuugcgagugccccgggaggucucguagaccgugcaccaugagcacgaauccuaaaccucaaagaaaaaccaaacguaac

SEQ ID NO: 79 - RNA, 383 nt, Hepatitis C virus
  HCV G266G267G268->C266C267C268(d) IRES
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguucccucgcgaaaggccuugugguacugccugauagggugcuugcgagugccccgggaggucucguagaccgugcaccaugagcacgaauccuaaaccucaaagaaaaaccaaacguaac

SEQ ID NO: 80 - RNA, 383 nt, Hepatitis C virus
  HCV G266->A/G268->T(d) IRES
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguuaguucgcgaaaggccuugugguacugccugauagggugcuugcgagugccccgggaggucucguagaccgugcaccaugagcacgaauccuaaaccucaaagaaaaaccaaacguaac

SEQ ID NO: 81 - RNA, 383 nt, Hepatitis C virus
  HVC IIIa->IIIa-comp; AGTA->TCAT(d) IRES
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugucaucaccggaauugccaggacgaccggguccuuucuuggauaaacccgcucaaugccuggagauuugggcgugcccccgcaagacugcuagccgaguaguguugggucgcgaaaggccuugugguacugccugauagggugcuugcgagugccccgggaggucucguagaccgugcaccaugagcacgaauccuaaaccucaaagaaaaaccaaacguaac

SEQ ID NO: 82 - RNA, 383 nt, Hepatitis C virus
  HCV IIIe-IIIe-comp; TGATAG->ACTATC(d) IRES
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguugggucgcgaaaggccuugugguacugccacuaucggugcuugcgagugccccgggaggucucguagaccgugcaccaugagcacgaauccuaaaccucaaagaaaaaccaaacguaac

SEQ ID NO: 83 - RNA, 1190 nt, Artificial Sequence
  SINEUP-FXN in antisense orientation to FXN mRNA isoform 1; BD= -40/+32 relative to M1-AUG
ccggcuacugcgcggcgcccgagaguccacaugcugcuccgggucugccgcccgcuccgcccuccagcgcugaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaaaaaacaaacaaacgaacgaacagcaagggagcuggguaugacaacacauacuauaauucuaguacucaggaugcugaaacaggaggauugccugacugggagauauaaggagaaucuguugucacccccaccccuccccauaaaggcagaauaaaagaacguccuauaaacaaauaaacaaacaacccaauaaaacaaaaccaagaucucuccaccuuuucuuugcuuuuucagacuuuguaauaaggcccuuuggagugcaggauauucggcaggacaagcagagagggagaccaucaguucuuucuuugaucaagaagacuauguuccuuagcaaacugguguguauuaucucuuaugcaaugagccuggaaagagggcacagccaccgaggaugguacagcauggauggaugguacgcuacagagacucgggagcccaacugugaguggcugacuggcaugguagguucagggaagaauuggccugugaagaaaauguucuugaaaagugaacaaggugcaggagguaggaguggguccugggcaaagcagggggugcaucccagccucagggaauagcacagcagaggucuguugaugcaugcgagugcaugaccugcuugccaauagacgaucaagaaugggcaaagcaucaugggugaugagugggagaggggaugagacauuccuuucucccugcugagacuuccauugaaccgaugaguucugaauagaagaugcccccccacccccccaccaguguagaaucugaagggaggcauauauuacccuauauuacucuguguuggcggcgagcuaucugacagccaaccuucccauacauuucauugggcauacacuaaugacaggaaguuccuuuugcuuguaugcaagagauggcucacacgauggagaauuuaaucuug

SEQ ID NO: 84 - RNA, 1162 nt, Artificial Sequence
  SINEUP-FXN in antisense orientation to FXN mRNA isoform 1; BD= -40/+4 relative to M1-AUG
acaugcugcuccgggucugccgcccgcuccgcccuccagcgcugaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaaaaaacaaacaaacgaacgaacagcaagggagcuggguaugacaacacauacuauaauucuaguacucaggaugcugaaacaggaggauugccugacugggagauauaaggagaaucuguugucacccccaccccuccccauaaaggcagaauaaaagaacguccuauaaacaaauaaacaaacaacccaauaaaacaaaaccaagaucucuccaccuuuucuuugcuuuuucagacuuuguaauaaggcccuuuggagugcaggauauucggcaggacaagcagagagggagaccaucaguucuuucuuugaucaagaagacuauguuccuuagcaaacugguguguauuaucucuuaugcaaugagccuggaaagagggcacagccaccgaggaugguacagcauggauggaugguacgcuacagagacucgggagcccaacugugaguggcugacuggcaugguagguucagggaagaauuggccugugaagaaaauguucuugaaaagugaacaaggugcaggagguaggaguggguccugggcaaagcagggggugcaucccagccucagggaauagcacagcagaggucuguugaugcaugcgagugcaugaccugcuugccaauagacgaucaagaaugggcaaagcaucaugggugaugagugggagaggggaugagacauuccuuucucccugcugagacuuccauugaaccgaugaguucugaauagaagaugcccccccacccccccaccaguguagaaucugaagggaggcauauauuacccuauauuacucuguguuggcggcgagcuaucugacagccaaccuucccauacauuucauugggcauacacuaaugacaggaaguuccuuuugcuuguaugcaagagauggcucacacgauggagaauuuaaucuug

SEQ ID NO: 85 - RNA, 1171 nt, Artificial Sequence
  SINEUP-FXN in antisense orientation to FXN mRNA isoform 1; BD= -21/+32 relative to M1-AUG
ccggcuacugcgcggcgcccgagaguccacaugcugcuccgggucugccgcccaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaaaaaacaaacaaacgaacgaacagcaagggagcuggguaugacaacacauacuauaauucuaguacucaggaugcugaaacaggaggauugccugacugggagauauaaggagaaucuguugucacccccaccccuccccauaaaggcagaauaaaagaacguccuauaaacaaauaaacaaacaacccaauaaaacaaaaccaagaucucuccaccuuuucuuugcuuuuucagacuuuguaauaaggcccuuuggagugcaggauauucggcaggacaagcagagagggagaccaucaguucuuucuuugaucaagaagacuauguuccuuagcaaacugguguguauuaucucuuaugcaaugagccuggaaagagggcacagccaccgaggaugguacagcauggauggaugguacgcuacagagacucgggagcccaacugugaguggcugacuggcaugguagguucagggaagaauuggccugugaagaaaauguucuugaaaagugaacaaggugcaggagguaggaguggguccugggcaaagcagggggugcaucccagccucagggaauagcacagcagaggucuguugaugcaugcgagugcaugaccugcuugccaauagacgaucaagaaugggcaaagcaucaugggugaugagugggagaggggaugagacauuccuuucucccugcugagacuuccauugaaccgaugaguucugaauagaagaugcccccccacccccccaccaguguagaaucugaagggaggcauauauuacccuauauuacucuguguuggcggcgagcuaucugacagccaaccuucccauacauuucauugggcauacacuaaugacaggaaguuccuuuugcuuguaugcaagagauggcucacacgauggagaauuuaaucuug

SEQ ID NO: 86 - RNA, 1143 nt, Artificial Sequence
  SINEUP-FXN in antisense orientation to FXN mRNA isoform 1; BD= -21/+4 relative to M1-AUG
acaugcugcuccgggucugccgcccaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaaaaaacaaacaaacgaacgaacagcaagggagcuggguaugacaacacauacuauaauucuaguacucaggaugcugaaacaggaggauugccugacugggagauauaaggagaaucuguugucacccccaccccuccccauaaaggcagaauaaaagaacguccuauaaacaaauaaacaaacaacccaauaaaacaaaaccaagaucucuccaccuuuucuuugcuuuuucagacuuuguaauaaggcccuuuggagugcaggauauucggcaggacaagcagagagggagaccaucaguucuuucuuugaucaagaagacuauguuccuuagcaaacugguguguauuaucucuuaugcaaugagccuggaaagagggcacagccaccgaggaugguacagcauggauggaugguacgcuacagagacucgggagcccaacugugaguggcugacuggcaugguagguucagggaagaauuggccugugaagaaaauguucuugaaaagugaacaaggugcaggagguaggaguggguccugggcaaagcagggggugcaucccagccucagggaauagcacagcagaggucuguugaugcaugcgagugcaugaccugcuugccaauagacgaucaagaaugggcaaagcaucaugggugaugagugggagaggggaugagacauuccuuucucccugcugagacuuccauugaaccgaugaguucugaauagaagaugcccccccacccccccaccaguguagaaucugaagggaggcauauauuacccuauauuacucuguguuggcggcgagcuaucugacagccaaccuucccauacauuucauugggcauacacuaaugacaggaaguuccuuuugcuuguaugcaagagauggcucacacgauggagaauuuaaucuug

SEQ ID NO: 87 - RNA, 1158 nt, Artificial Sequence
  SINEUP-FXN in antisense orientation to FXN mRNA isoform 1; BD= -40/+0 relative to M1-AUG
gcugcuccgggucugccgcccgcuccgcccuccagcgcugaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaaaaaacaaacaaacgaacgaacagcaagggagcuggguaugacaacacauacuauaauucuaguacucaggaugcugaaacaggaggauugccugacugggagauauaaggagaaucuguugucacccccaccccuccccauaaaggcagaauaaaagaacguccuauaaacaaauaaacaaacaacccaauaaaacaaaaccaagaucucuccaccuuuucuuugcuuuuucagacuuuguaauaaggcccuuuggagugcaggauauucggcaggacaagcagagagggagaccaucaguucuuucuuugaucaagaagacuauguuccuuagcaaacugguguguauuaucucuuaugcaaugagccuggaaagagggcacagccaccgaggaugguacagcauggauggaugguacgcuacagagacucgggagcccaacugugaguggcugacuggcaugguagguucagggaagaauuggccugugaagaaaauguucuugaaaagugaacaaggugcaggagguaggaguggguccugggcaaagcagggggugcaucccagccucagggaauagcacagcagaggucuguugaugcaugcgagugcaugaccugcuugccaauagacgaucaagaaugggcaaagcaucaugggugaugagugggagaggggaugagacauuccuuucucccugcugagacuuccauugaaccgaugaguucugaauagaagaugcccccccacccccccaccaguguagaaucugaagggaggcauauauuacccuauauuacucuguguuggcggcgagcuaucugacagccaaccuucccauacauuucauugggcauacacuaaugacaggaaguuccuuuugcuuguaugcaagagauggcucacacgauggagaauuuaaucuug

SEQ ID NO: 88 - RNA, 1132 nt, Artificial Sequence
  SINEUP-FXN in antisense orientation to FXN mRNA isoform 1; BD= -14/+0 relative to M1-AUG
gcugcuccgggucuaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaaaaaacaaacaaacgaacgaacagcaagggagcuggguaugacaacacauacuauaauucuaguacucaggaugcugaaacaggaggauugccugacugggagauauaaggagaaucuguugucacccccaccccuccccauaaaggcagaauaaaagaacguccuauaaacaaauaaacaaacaacccaauaaaacaaaaccaagaucucuccaccuuuucuuugcuuuuucagacuuuguaauaaggcccuuuggagugcaggauauucggcaggacaagcagagagggagaccaucaguucuuucuuugaucaagaagacuauguuccuuagcaaacugguguguauuaucucuuaugcaaugagccuggaaagagggcacagccaccgaggaugguacagcauggauggaugguacgcuacagagacucgggagcccaacugugaguggcugacuggcaugguagguucagggaagaauuggccugugaagaaaauguucuugaaaagugaacaaggugcaggagguaggaguggguccugggcaaagcagggggugcaucccagccucagggaauagcacagcagaggucuguugaugcaugcgagugcaugaccugcuugccaauagacgaucaagaaugggcaaagcaucaugggugaugagugggagaggggaugagacauuccuuucucccugcugagacuuccauugaaccgaugaguucugaauagaagaugcccccccacccccccaccaguguagaaucugaagggaggcauauauuacccuauauuacucuguguuggcggcgagcuaucugacagccaaccuucccauacauuucauugggcauacacuaaugacaggaaguuccuuuugcuuguaugcaagagauggcucacacgauggagaauuuaaucuug

SEQ ID NO: 89 - RNA, 1136 nt, Artificial Sequence
  SINEUP-FXN in antisense orientation to FXN mRNA isoform 1; BD= -14/+4 relative to M1-AUG
acaugcugcuccgggucuaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaaaaaacaaacaaacgaacgaacagcaagggagcuggguaugacaacacauacuauaauucuaguacucaggaugcugaaacaggaggauugccugacugggagauauaaggagaaucuguugucacccccaccccuccccauaaaggcagaauaaaagaacguccuauaaacaaauaaacaaacaacccaauaaaacaaaaccaagaucucuccaccuuuucuuugcuuuuucagacuuuguaauaaggcccuuuggagugcaggauauucggcaggacaagcagagagggagaccaucaguucuuucuuugaucaagaagacuauguuccuuagcaaacugguguguauuaucucuuaugcaaugagccuggaaagagggcacagccaccgaggaugguacagcauggauggaugguacgcuacagagacucgggagcccaacugugaguggcugacuggcaugguagguucagggaagaauuggccugugaagaaaauguucuugaaaagugaacaaggugcaggagguaggaguggguccugggcaaagcagggggugcaucccagccucagggaauagcacagcagaggucuguugaugcaugcgagugcaugaccugcuugccaauagacgaucaagaaugggcaaagcaucaugggugaugagugggagaggggaugagacauuccuuucucccugcugagacuuccauugaaccgaugaguucugaauagaagaugcccccccacccccccaccaguguagaaucugaagggaggcauauauuacccuauauuacucuguguuggcggcgagcuaucugacagccaaccuucccauacauuucauugggcauacacuaaugacaggaaguuccuuuugcuuguaugcaagagauggcucacacgauggagaauuuaaucuug

SEQ ID NO: 90 - RNA, 1162 nt, Artificial Sequence
  SINEUP-FXN in antisense orientation to FXN mRNA isoform 1; BD= -40/+4 relative to M76-AUG
ucaucaaauagacacucugcuuuuugacauuccaaaucugguugaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaaaaaacaaacaaacgaacgaacagcaagggagcuggguaugacaacacauacuauaauucuaguacucaggaugcugaaacaggaggauugccugacugggagauauaaggagaaucuguugucacccccaccccuccccauaaaggcagaauaaaagaacguccuauaaacaaauaaacaaacaacccaauaaaacaaaaccaagaucucuccaccuuuucuuugcuuuuucagacuuuguaauaaggcccuuuggagugcaggauauucggcaggacaagcagagagggagaccaucaguucuuucuuugaucaagaagacuauguuccuuagcaaacugguguguauuaucucuuaugcaaugagccuggaaagagggcacagccaccgaggaugguacagcauggauggaugguacgcuacagagacucgggagcccaacugugaguggcugacuggcaugguagguucagggaagaauuggccugugaagaaaauguucuugaaaagugaacaaggugcaggagguaggaguggguccugggcaaagcagggggugcaucccagccucagggaauagcacagcagaggucuguugaugcaugcgagugcaugaccugcuugccaauagacgaucaagaaugggcaaagcaucaugggugaugagugggagaggggaugagacauuccuuucucccugcugagacuuccauugaaccgaugaguucugaauagaagaugcccccccacccccccaccaguguagaaucugaagggaggcauauauuacccuauauuacucuguguuggcggcgagcuaucugacagccaaccuucccauacauuucauugggcauacacuaaugacaggaaguuccuuuugcuuguaugcaagagauggcucacacgauggagaauuuaaucuug

SEQ ID NO: 91 - RNA, 1158 nt, Artificial Sequence
  SINEUP-FXN in antisense orientation to FXN mRNA isoform 1; BD= -40/+0 relative to M76-AUG
caaauagacacucugcuuuuugacauuccaaaucugguugaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaaaaaacaaacaaacgaacgaacagcaagggagcuggguaugacaacacauacuauaauucuaguacucaggaugcugaaacaggaggauugccugacugggagauauaaggagaaucuguugucacccccaccccuccccauaaaggcagaauaaaagaacguccuauaaacaaauaaacaaacaacccaauaaaacaaaaccaagaucucuccaccuuuucuuugcuuuuucagacuuuguaauaaggcccuuuggagugcaggauauucggcaggacaagcagagagggagaccaucaguucuuucuuugaucaagaagacuauguuccuuagcaaacugguguguauuaucucuuaugcaaugagccuggaaagagggcacagccaccgaggaugguacagcauggauggaugguacgcuacagagacucgggagcccaacugugaguggcugacuggcaugguagguucagggaagaauuggccugugaagaaaauguucuugaaaagugaacaaggugcaggagguaggaguggguccugggcaaagcagggggugcaucccagccucagggaauagcacagcagaggucuguugaugcaugcgagugcaugaccugcuugccaauagacgaucaagaaugggcaaagcaucaugggugaugagugggagaggggaugagacauuccuuucucccugcugagacuuccauugaaccgaugaguucugaauagaagaugcccccccacccccccaccaguguagaaucugaagggaggcauauauuacccuauauuacucuguguuggcggcgagcuaucugacagccaaccuucccauacauuucauugggcauacacuaaugacaggaaguuccuuuugcuuguaugcaagagauggcucacacgauggagaauuuaaucuug

SEQ ID NO: 92 - RNA, 1188 nt, Artificial Sequence
  SINEUP-FXN in antisense orientation to FXN mRNA isoform 1; BD= -70/+0 relative to M76-AUG
caaauagacacucugcuuuuugacauuccaaaucugguugaggccacguugguucgaacuugcgcggcggaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcuccagucucuuaaaaaacaaacaaacgaacgaacagcaagggagcuggguaugacaacacauacuauaauucuaguacucaggaugcugaaacaggaggauugccugacugggagauauaaggagaaucuguugucacccccaccccuccccauaaaggcagaauaaaagaacguccuauaaacaaauaaacaaacaacccaauaaaacaaaaccaagaucucuccaccuuuucuuugcuuuuucagacuuuguaauaaggcccuuuggagugcaggauauucggcaggacaagcagagagggagaccaucaguucuuucuuugaucaagaagacuauguuccuuagcaaacugguguguauuaucucuuaugcaaugagccuggaaagagggcacagccaccgaggaugguacagcauggauggaugguacgcuacagagacucgggagcccaacugugaguggcugacuggcaugguagguucagggaagaauuggccugugaagaaaauguucuugaaaagugaacaaggugcaggagguaggaguggguccugggcaaagcagggggugcaucccagccucagggaauagcacagcagaggucuguugaugcaugcgagugcaugaccugcuugccaauagacgaucaagaaugggcaaagcaucaugggugaugagugggagaggggaugagacauuccuuucucccugcugagacuuccauugaaccgaugaguucugaauagaagaugcccccccacccccccaccaguguagaaucugaagggaggcauauauuacccuauauuacucuguguuggcggcgagcuaucugacagccaaccuucccauacauuucauugggcauacacuaaugacaggaaguuccuuuugcuuguaugcaagagauggcucacacgauggagaauuuaaucuug

SEQ ID NO: 93 - RNA, 220 nt, Artificial Sequence
  miniSINEUP-FXN in antisense orientation to FXN mRNA; BD= -40/+0 relative to M1-AUG
gcugcuccgggucugccgcccgcuccgcccuccagcgcugaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcucc

SEQ ID NO: 94 - RNA, 194 nt, Artificial Sequence
  miniSINEUP-FXN in antisense orientation to FXN mRNA; BD= -14/+0 relative to M1-AUG
gcugcuccgggucuaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcucc

SEQ ID NO: 95 - RNA, 198 nt, Artificial Sequence
  miniSINEUP-FXN in antisense orientation to FXN mRNA; BD= -14/+4 relative to M1-AUG
acaugcugcuccgggucuaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcucc

SEQ ID NO: 96 - RNA, 224 nt, Artificial Sequence
  miniSINEUP-FXN in antisense orientation to FXN mRNA; BD= -40/+4 relative to M76-AUG
ucaucaaauagacacucugcuuuuugacauuccaaaucugguugaucugcagaauuccagugcuagaggaggucagaagagggcauuggaucccccagaacuggaguuauacgguaaccucguggugguugugaaccaccauguggauggauauugaguuccaaacacugguccugugcaagagcauccagugcucuuaagugcugagccaucucuuuagcucc

SEQ ID NO: 97 - RNA, 136 nt, Artificial Sequence
  microSINEUP-FXN in antisense orientation to FXN mRNA; BD= -40/+0 relative to M1-AUG
gcugcuccgggucugccgcccgcuccgcccuccagcgcugaucugcagaauucgcccuugaacuggaguuauacgguaaccucguggugguugugaaccaccauguggauggauauugaguuccaaacacuggucc

SEQ ID NO: 98 - RNA, 110 nt, Artificial Sequence
  microSINEUP-FXN in antisense orientation to FXN mRNA; BD= -14/+0 relative to M1-AUG
gcugcuccgggucuaucugcagaauucgcccuugaacuggaguuauacgguaaccucguggugguugugaaccaccauguggauggauauugaguuccaaacacuggucc

(19) United States
(12) Patent Application Publication
(10) Pub. No.: US 2022/0259595 A1
(43) Pub. Date: Aug. 18, 2022

(54) FUNCTIONAL NUCLEIC ACID MOLECULES UPREGULATING THE TRANSLATION OF A FRATAXIN MRNA

(71) Applicant: Scuola Internazionale Superiore Studi Internazionale Superiore Di Studi Avanzati - SISSA, Trieste (IT)

(72) Inventors: Stefano Gustincich, Trieste (IT); Silvia Zucchelli (deceases), (US); Carlotta Bon, Trieste (IT)

(21) Appl. No.: 17/625,433

(22) PCT Filed: Jul. 10, 2020
(86) PCT No.: PCT/EP2020/069519

§ 371 Date: Jan. 7, 2022

(30) Foreign Application Priority Data
Jul. 11, 2019 (IT) 102019000011490

(51) Int. Cl. C12N 15/113; C12N 5/071; C12N 15/85

(52) U.S. Cl. CPC C12N 15/113; C12N 5/0687; C12N 15/85; C12N 2310/531; C12N 2800/107; C12N 2840/203; C12N 2830/002; C12N 2740/15043; C12N 2750/14143; C12N 2310/11

(57) ABSTRACT

There are disclosed functional nucleic acid molecules comprising at least one target binding sequence comprising a sequence reverse complementary to a frataxin mRNA sequence; and a regulatory sequence comprising a SINE B2 element or a functionally active fragment of a SINE B2 element or an internal ribosome entry site (IRES) sequence or an IRES derived sequence.

### TECHNICAL FIELD OF THE INVENTION

The present invention relates to functional nucleic acid molecules comprising at least one target binding sequence comprising a sequence reverse complementary to a frataxin mRNA sequence; and a regulatory sequence comprising an RNA comprising a SINE B2 element or a functionally active fragment of a SINE B2 element or an internal ribosome entry site (IRES) sequence or an IRES derived sequence.

### BACKGROUND

Friedreich's ataxia (FRDA) is a life-threatening monogenic disease with neuro- and cardio-degenerative progression. It represents the most frequent type of inherited ataxia, affecting more than 15,000 patients in Western countries. Patients typically show degeneration of large sensory neurons of the dorsal root ganglia, Betz pyramidal neurons of the cerebral cortex and lateral cortico-spinal and spinocerebellar tracts, as well as lesions in the dentate nucleus of the cerebellum. In addition, non-neurological degeneration causes hypertrophic cardiomyopathy and increased incidence of diabetes mellitus. Neurodegenerative motor symptoms typically appear before adolescence with progressive gait instability and loss of coordination, while the cardiac component of the disease causes premature mortality at a mean age of 40 years. Almost all FRDA patients carry an intronic homozygous expansion of natural GAA repeats located in the FXN gene. The human FXN locus contains normally from 10 to 66 GAA-triplet repeats within the first intron, whereas FRDA individuals have an hyperexpansion of such repeats, up to 1700 triplets. In a small percentage of cases, however, patients are compound heterozygotes for GAA expansion on one FXN allele and a second allele with a small insertion, deletion or point mutation in FXN open reading frame. Longer hyperexpansions result in a more severe phenotype with an earlier onset and faster progression. GAA repeat expansions impair FXN transcription by inducing the formation of triple helical DNA structures (sticky DNA), persistent DNA/RNA hybrids (R-loops) and specific epigenetic modifications. The FXN gene encodes for the precursor of frataxin, a small iron-binding protein, that is mainly, but not exclusively, confined inside the mitochondrial matrix, where it is converted into the functional mature form. Although its primary function is still debated, mature frataxin is a key component of the iron-sulfur cluster (ISC) biosynthetic apparatus, which provides the essential cofactor to all ISC-dependent enzymes of the cell. As a consequence of insufficient FXN expression, defective ISC biosynthesis triggers a series of vicious cycles leading to deregulated intracellular iron homeostasis, impaired mitochondrial electron transport chain and higher sensitivity to trigger oxidant- and stress-induced cell death. Currently, there are no therapies to treat the disease or prevent its progression.

The most promising approaches point to restore sufficient frataxin levels, mostly by enhancing FXN transcription. Among them, IFN-γ and dyclonine have been identified as encouraging candidates by drug repositioning programs. Synthetic histone deacetylase (HDAC) inhibitors have been described to increase FXN mRNA in FRDA-derived cells and in FRDA animal models. More recently, synthetic nucleic acids were successfully employed targeting GAA repeats, acting as R-loops inhibitors. Moreover, polyamide-based transcription factors capable of binding GAA microsatellite were developed. Interestingly, protein replacement therapy, based on TAT-frataxin delivery, and frataxin degradation prevention, by a class of ubiquitin-competing small molecules, have recently been proposed as potential treatments targeting the frataxin polypeptide. Finally, an effective gene replacement strategy in the FRDA mouse model opened new opportunities for gene therapy in the future.

However, recent data has proved that prolonged over-expression at non-physiological levels of frataxin affects the cellular metabolism, leading to a significant increase of oxidative stress and labile iron pool levels. These cellular alterations are similar to those observed when the gene is partly silenced, as occurs in FRDA patients. These results suggest that any long-term therapeutic intervention must finely tune frataxin protein levels within a physiological range.

In view of the above, there is a need for new therapeutic approaches for Friedreich's ataxia, in particular new therapies that do not increase frataxin protein beyond physiological levels. Furthermore, there is a need for new therapies that target frataxin expression in a highly gene-specific manner limiting side effects.

Recently, a novel functional class of antisense (AS) lncRNAs was identified, which increase translation of partially overlapping sense protein-coding mRNAs. These RNAs are also called SINEUPs, as they require a SINE B2 element to UP-regulate translation and are disclosed in WO 2012/133947.

AS Uchl1, a lncRNA antisense to the mouse orthologue of human Uchl1/PARK5 gene, can be considered the representative member of this new class of lncRNAs, as it was found to increase UchL1 protein synthesis acting at a post-transcriptional level. AS Uchl1 activity depends on the combination of two functional domains: at the 5′ end, the overlapping region, indicated as “binding domain” or “target binding sequence”, dictates AS Uchl1 specificity towards Uchl1 mRNA; at the 3′ end, the non-overlapping region contains an embedded inverted SINE B2 element, which acts as “effector domain” (or “regulatory sequence”) and triggers translation up-regulation of bound target mRNA.

More than 30 antisense lncRNAs promote translation up-regulation of partially overlapping mRNAs. By replacing the binding domain, it is possible to re-direct AS Uchl1 activity towards a target mRNA of choice.

### SUMMARY OF THE INVENTION

It is an object of the present invention to provide a functional nucleic acid molecule that increases the frataxin protein without overcoming physiological levels, targets frataxin expression in a highly gene-specific manner and limits side effects.

This object is achieved by means of the functional nucleic acid molecule as defined herein.

Other objects of the present invention are to provide a DNA molecule encoding the functional nucleic acid molecule, a composition, and uses as defined herein.

### Definitions

By “functional nucleic acid molecule” there is intended generally that the nucleic acid molecule is capable of enhancing the translation of a target mRNA of interest, in this particular case a frataxin mRNA.

By “frataxin mRNA sequence” there is intended an mRNA sequence of any length of at least 10 nucleotides comprised in the mRNA of the corresponding frataxin (FXN) gene. The FXN gene sequence is known in the art, for example see Gene ID: 2395 or Ensembl ID: ENSG00000165060. The FXN gene encodes the frataxin protein. The frataxin protein sequence is known in the art, for example see UniProt ID: Q16595.

The term “SINE” (Short Interspersed Nuclear Element) may be referred to as a non-LTR (long terminal repeat) retrotransposon, and is an interspersed repetitive sequence (a) which encodes a protein having neither reverse-transcription activity nor endonuclease activity or the like and (b) whose complete or incomplete copy sequences exist abundantly in genomes of living organisms.

The term “SINE B2 element” is defined in WO 2012/133947, where specific examples are also provided (see table starting on page 69 of the PCT publication). The term is intended to encompass both SINE B2 elements in direct orientation and in inverted orientation relative to the 5′ to 3′ orientation of the functional nucleic acid molecule. SINE B2 elements may be identified, for example, using programs like RepeatMask as published (Bedell et al. Bioinformatics. 2000 November; 16(11): 1040-1. MaskerAid: a performance enhancement to RepeatMasker). A sequence may be recognizable as a SINE B2 element by returning a hit in a Repbase database with respect to a consensus sequence of a SINE B2, with a Smith-Waterman (SW) score of over 225, which is the default cutoff in the RepeatMasker program. Generally a SINE B2 element is not less than 20 bp and not more than 400 bp. Preferably, the SINE B2 is derived from tRNA.

By the term “functionally active fragment of a SINE B2 element” there is intended a portion of sequence of a SINE B2 element that retains protein translation enhancing efficiency. This term also includes sequences which are mutated in one or more nucleotides with respect to the wild-type sequences, but retain protein translation enhancing efficiency. The term is intended to encompass both SINE B2 elements in direct orientation and in inverted orientation relative to the 5′ to 3′ orientation of the functional nucleic acid molecule.

The terms “internal ribosome entry site (IRES) sequence” and “internal ribosome entry site (IRES) derived sequence” are defined in WO 2019/058304. IRES sequences recruit the 40S ribosomal subunit and promote cap-independent translation of a subset of protein coding mRNAs. IRES sequences are generally found in the 5′ untranslated region of cellular mRNAs coding for stress-response genes, thus stimulating their translation in cis. It will be understood by the term “IRES derived sequence” there is intended a sequence of nucleic acid with a homology to an IRES sequence so as to retain the functional activity thereof, i.e. a translation enhancing activity. In particular, the IRES derived sequence can be obtained from a naturally occurring IRES sequence by genetic engineering or chemical modification, e.g. by isolating a specific sequence of the IRES sequence which remains functional, or mutating/deleting/introducing one or more nucleotides in the IRES sequence, or replacing one or more nucleotides in the IRES sequence with structurally modified nucleotides or analogs. More in particular, the skilled in the art would know that an IRES derived sequence is a nucleotide sequence capable of promoting translation of a second cistron in a bicistronic construct. Typically, a dual luciferase (Firefly luciferase, Renilla Luciferase) encoding plasmid is used for experimental tests. A major database exists, namely IRESite, for the annotation of nucleotide sequences that have been experimentally validated as IRES, using dual reporter or bicistronic assays (http://iresite.org/IRESite_web.php). Within the IRESite, a web-based tool is available to search for sequence-based and structure-based similarities between a query sequence of interest and the entirety of annotated and experimentally validated IRES sequences within the database. The output of the program is a probability score for any nucleotide sequence to be able to act as IRES in a validation experiment with bicistronic constructs. Additional sequence-based and structure-based web-based browsing tools are available to suggest, with a numerical predicting value, the IRES activity potentials of any given nucleotide sequence (http://rna.informatik.uni-freiburg.de/; http://regrna.mbc.nctu.edu.tw/index1.php).

By the term “miniSINEUP” there is intended a nucleic acid molecule consisting of a binding domain (complementary sequence to target mRNA), a spacer sequence, and any SINE or SINE-derived sequence or IRES-derived sequence as the effector domain (Zucchelli et al., Front Cell Neurosci., 9: 174, 2015).

By the term “microSINEUP” there is intended a nucleic acid molecule consisting of a binding domain (complementary sequence to target mRNA), a spacer sequence, and a functionally active fragment of the SINE or SINE-derived sequence or IRES-derived sequence. For example, the functionally active fragment may be a 77 bp sequence corresponding to nucleotides 44 to 120 of the 167 bp SINE B2 element in AS Uchl1.

Polypeptide or polynucleotide sequences are said to be the same as or “identical” to other polypeptide or polynucleotide sequences, if they share 100% sequence identity over their entire length. Residues in sequences are numbered from left to right, i.e. from N- to C-terminus for polypeptides; from 5′ to 3′ terminus for polynucleotides.

For the purposes of comparing two closely-related polynucleotide sequences, the “% sequence identity” between a first nucleotide sequence and a second nucleotide sequence may be calculated using NCBI BLAST, using standard settings for nucleotide sequences (BLASTN). For the purposes of comparing two closely-related polypeptide sequences, the “% sequence identity” between a first polypeptide sequence and a second polypeptide sequence may be calculated using NCBI BLAST, using standard settings for polypeptide sequences (BLASTP). A “difference” between sequences refers to an insertion, deletion or substitution of a single nucleotide in a position of the second sequence, compared to the first sequence. Two sequences can contain one, two or more such differences. Insertions, deletions or substitutions in a second sequence which is otherwise identical (100% sequence identity) to a first sequence result in reduced % sequence identity.

### BRIEF DESCRIPTION OF THE DRAWINGS

FIG. 1 shows a schematic representation of SINEUP functional domains and of the human FXN gene with examples of the target binding domains of the functional nucleic acid according to the invention.

FIG. 2 shows that synthetic SINEUPs increase endogenous frataxin protein level in human cells in vitro.

FIG. 3 shows SINEUP effect on FXN knockdown HEK293T/17 cells.

FIG. 4 shows that miniSINEUPs increase endogenous frataxin protein level in human cells in vitro.

FIG. 5 shows that miniSINEUPs increase endogenous frataxin protein level in SH-SY5Y cells in vitro.

FIG. 6 shows that the binding domain is specific and that frataxin protein expression in vitro increases selectively.

FIG. 7 shows effector domain optimization.

FIG. 8 shows miniSINEUPs lentiviral transduction optimization.

FIG. 9 shows lentiviral infection of HEK 293T/17 cells.

FIG. 10 shows increased endogenous FXN protein expression in FRDA-derived fibroblasts.

FIG. 11 shows that AAV9-miniSINEUPs increase endogenous frataxin protein level in HEK 293T/17 cells in vitro.

FIG. 12 shows protein rescue of FRDA-derived lymphoblasts.

FIG. 13 shows the phenotypic rescue of FRDA-derived lymphoblasts.

FIG. 14 shows the miniSINEUP-FXN effect on off-target protein expression.

### DETAILED DESCRIPTION OF THE INVENTION

A functional nucleic acid molecule of the present invention comprises at least one target binding sequence comprising a sequence reverse complementary to a frataxin mRNA sequence and at least one regulatory sequence comprising an RNA comprising a SINE B2 element or a functionally active fragment of a SINE B2 element or an internal ribosome entry site (IRES) sequence or an IRES derived sequence.

The functional nucleic acid molecules of the invention are able to modulate protein translation of the mRNA target however, compared to other methods, the modulation is not the result of modifying the target gene and therefore does not include the risks associated with genome editing. Furthermore, the functional nucleic acid molecules are highly specific to the target, reducing any off-target side effects.

Regulatory Sequences

In one embodiment, the regulatory sequence has protein translation enhancing efficiency. The increase of the protein translation efficiency indicates that the efficiency is increased as compared to a case where the functional nucleic acid molecule according to the present invention is not present in a system. In one embodiment, expression of the protein encoded by the target mRNA is increased by at least 1.5 fold, such as at least 2 fold. In a further embodiment, expression of the protein encoded by the target mRNA is increased between 1.2 to 3 fold, such as between 1.5 and 2.2 fold.

In one embodiment, the regulatory sequence is located 3′ of the target binding sequence. The regulatory sequence may be in a direct or inverted orientation relative to the 5′ to 3′ orientation of the functional nucleic acid molecule. Reference to “direct” refers to the situation in which the regulatory sequence is embedded (inserted) with the same 5′ to 3′ orientation as the functional nucleic acid molecule. Instead, “inverted” refers to the situation in which the regulatory sequence is 3′ to 5′ oriented relative to the functional nucleic acid molecule.

Preferably, the at least one regulatory sequence comprises a sequence with at least 75% sequence identity, preferably at least 90% sequence identity, more preferably at least 95% sequence identity, even more preferably 100% sequence identity with a sequence selected from the group consisting of SEQ ID NO: 1-82. In one embodiment, the at least one regulatory sequence consists of a sequence with at least 75% sequence identity, preferably at least 90% sequence identity, more preferably at least 95% sequence identity, even more preferably 100% sequence identity with a sequence selected from the group consisting of SEQ ID NO: 1-82.

In one embodiment, the regulatory sequence comprises a SINE B2 element or a functionally active fragment of a SINE B2 element. The SINE B2 element is preferably in an inverted orientation relative to the 5′ to 3′ orientation of the functional nucleic acid molecule, i.e. an inverted SINE B2 element. As mentioned in the definitions section, inverted SINE B2 elements are disclosed and exemplified in WO 2012/133947.

Preferably, the at least one regulatory sequence comprises a sequence with at least 75% sequence identity, preferably 90% sequence identity, more preferably 95% sequence identity, even more preferably 100% sequence identity with a sequence selected from the group consisting of SEQ ID NO:1 to SEQ ID NO:51.

SEQ ID NO:1 (the inverted SINE B2 element in AS Uchl1) and SEQ ID NO:2 (the 77 nucleotide variant of the inverted SINE B2 element in AS Uchl1 that includes nucleotides 44 to 120) are particularly preferred.

Other inverted SINE B2 elements and functionally active fragments of inverted SINE B2 elements are SEQ ID NO:3 to SEQ ID NO:51. Experimental data showing the protein translation enhancing efficiency of these sequences is not explicitly shown in the present patent application, but is disclosed in a previous patent application in the name of the same applicant. SEQ ID NO:3 to SEQ ID NO:51 can therefore be used as regulatory sequences in molecules according to the present invention.

SEQ ID NO:3 to SEQ ID NO:6, SEQ ID NO:8 to SEQ ID NO:11, SEQ ID NO:18, SEQ ID NO:43 to SEQ ID NO:51 are functionally active fragments of inverted SINE B2 transposable element derived from AS Uchl1. The use of functional fragments reduces the size of the regulatory sequence which is advantageous if used in an expression vector (e.g. viral vectors which may be size-limited) because this provides more space for the target sequence and/or expression elements.

SEQ ID NO:7 is a full length 183 nt inverted SINE B2 transposable element derived from AS Uchl1. SEQ ID NO:12 to SEQ ID NO:17, SEQ ID NO:19 and SEQ ID NO:20, SEQ ID NO:39 to SEQ ID NO:42 are mutated functionally active fragments of inverted SINE B2 transposable element derived from AS Uchl1.

SEQ ID NO:21 to SEQ ID NO:25 and SEQ ID NO:28 to SEQ ID NO:38 are different SINE B2 transposable elements. SEQ ID NO:26 and SEQ ID NO:27 are sequences in which multiple inverted SINE B2 transposable element have been inserted.

Alternatively, the regulatory sequence comprises an IRES sequence or an IRES derived sequence. Therefore, in one embodiment, the regulatory sequence comprises an IRES sequence or an IRES derived sequence. Said sequence enhances translation of the target mRNA sequence.

Several IRESs having sequences ranging from 48 to 576 nucleotides have been tested with success, e.g. human Hepatitis C Virus (HCV) IRESs (e.g. SEQ ID NO: 53 and 54), human poliovirus IRESs (e.g. SEQ ID NO: 55 and 56), human encephalomyocarditis (EMCV) virus (e.g. SEQ ID NO: 57 and 58), human cricket paralysis (CrPV) virus (e.g. SEQ ID NO: 59 and 60), human Apaf-1 (e.g. SEQ ID NO: 61 and 62), human ELG-1 (e.g. SEQ ID NO: 63 and 64), human c-MYC (e.g. SEQ ID NO: 65-68), human dystrophin (DMD) (e.g. SEQ ID NO: 69 and 70).

Such sequences have been disclosed, defined and exemplified in WO 2019/058304. Preferably, such sequences have 75% sequence identity, preferably 90% sequence identity, more preferably 95% sequence identity, even more preferably 100% sequence identity to any of SEQ ID NO:53 to SEQ ID NO:82. More preferably, such sequences have 75% sequence identity, preferably 90% sequence identity, more preferably 95% sequence identity, even more preferably 100% sequence identity to any of SEQ ID NO:53 to SEQ ID NO:70.

Target Binding Sequences

In WO 2012/133947 it was already shown that the target binding sequence needs to have only about 60% similarity with a sequence reverse complementary to the target mRNA. As a matter of fact, the target binding sequence can even display a large number of mismatches and retain activity.

The target binding sequence comprises a sequence which is sufficient in length to bind to the frataxin mRNA transcript. Therefore, the target binding sequence may be at least 10 nucleotides long, such as at least 14 nucleotides long, such as least 18 nucleotides long. Furthermore, the target binding sequence may be less than 250 nucleotides long, preferably less than 200 nucleotides long, less than 150 nucleotides long, less than 100 nucleotides long, less than 80 nucleotides long, less than 60 nucleotides long or less than 50 nucleotides long. In one embodiment, the target binding sequence is between 4 and 50 nucleotides in length, such as between 18 and 44 nucleotides long.

The target binding sequence may be designed to hybridise with the 5′-untranslated region (5′ UTR) of the frataxin mRNA sequence. In one embodiment, the sequence is reverse complementary to 0 to 50 nucleotides, such as 0 to 40, 0 to 21 or 0 to 14 nucleotides of the 5′ UTR. Alternatively, or in combination, the target binding sequence may be designed to hybridise to the coding sequence (CDS) of the frataxin mRNA sequence. In one embodiment, the sequence is reverse complementary to 0 to 40 nucleotides, such as 0 to 32, 0 to 18 or 0 to 4 nucleotides of the CDS.

The target binding sequence may be designed to hybridise to a region upstream of an AUG site (start codon), such as a start codon within the CDS, of the frataxin mRNA sequence. In one embodiment, the sequence is reverse complementary to 0 to 80 nucleotides, such as 0 to 70 or 0 to 40 nucleotides of the AUG site. Alternatively, or in combination, the target binding sequence may be designed to hybridise to the frataxin mRNA sequence downstream of said AUG site. In one embodiment, the sequence is reverse complementary to 0 to 40 nucleotides, such as 0 to 4 nucleotides of the frataxin mRNA sequence downstream of said AUG site.

Preferably, the at least one target binding sequence is at least 10 nucleotides long and comprises, from 3′ to 5′:
1) a sequence reverse complementary to 0 to 50 nucleotides of the 5′ UTR and 0 to 40 nucleotides of the CDS of the frataxin mRNA sequence; or
2) a sequence reverse complementary to 0 to 80 nucleotides of the region upstream of an AUG site (start codon) of the frataxin mRNA and 0 to 40 nucleotides, preferably 0 to 10 nucleotides, of the CDS of the frataxin mRNA sequence downstream of said AUG site.

Of course in case 1) the coding sequence starts on the first AUG site (M1) of the mRNA. In case 2), the preferred AUG site is that corresponding to methionine 76 (M76) in exon 2.

More preferably, the at least one target binding sequence is at least 14 nucleotides long and comprises, from 3′ to 5′:
a sequence reverse complementary to 0 to 40 (preferably 0 to 21, more preferably 0 to 14) nucleotides of the 5′ UTR and 0 to 32 (preferably 0 to 4, more preferably 0) nucleotides of the CDS of the frataxin mRNA sequence; or
a sequence reverse complementary to 0 to 70 (preferably 0 to 40) nucleotides of the region upstream of an AUG site (start codon) of the frataxin mRNA and 0 to 4 (preferably 0) nucleotides of the CDS of the frataxin mRNA sequence downstream of said AUG site.

In one embodiment, the functional nucleic acid molecule comprises a sequence with at least 75% sequence identity, preferably at least 90% sequence identity, more preferably at least 95% sequence identity, even more preferably 100% sequence identity to any of SEQ ID NO: 83-98. In a further embodiment, the functional nucleic acid molecule consists of a sequence with at least 75% sequence identity, preferably at least 90% sequence identity, more preferably at least 95% sequence identity, even more preferably 100% sequence identity to any of SEQ ID NO: 83-98.

Structural Features

The functional nucleic acid molecule preferably comprises more than one regulatory sequence, which can be the same sequence repeated more than once, or a different regulatory sequence (i.e. a different SINE B2 element/functionally active fragment of a SINE B2 element/an IRES sequence/an IRES derived sequence).

The at least one target binding sequence and the at least one regulatory sequence are preferably connected by at least one spacer/linker sequence. In case of multiple sequences, several spacer/linker sequences can be inserted in-between the sequences. SEQ ID NO:52 is a non-limiting example of the spacer/linker sequence.

The functional nucleic acid molecule of the present invention is preferably a circular molecule. This conformation leads to a much more stable molecule that is degraded with greater difficulty within the cell (exonucleases cannot degrade circular molecules) and therefore remains active for a longer time.

Furthermore, the functional nucleic acid molecule may optionally comprise a non-coding 3′ tail sequence, which e.g. includes restriction sites useful for cloning the molecule in appropriate plasmids.

It should be noted that the functional nucleic acid molecules can enhance translation of the target gene of interest with no effects on mRNA quantities of the target gene. Therefore they can successfully be used as molecular tools to validate gene function in cells as well as to implement the pipelines of recombinant protein production.

DNA Molecules and Vectors

According to a further aspect of the invention, there is provided a DNA molecule encoding any of the above disclosed functional nucleic acid molecules. According to a further aspect of the invention, there is provided an expression vector comprising the above said DNA molecule.

Exemplary expression vectors are known in the art and may include, for example, plasmid vectors, viral vectors (for example adenovirus, adeno-associated virus, retrovirus or lentivirus vectors), phage vectors, cosmid vectors and the like. The choice of expression vector may be dependent upon the type of host cell to be used and the purpose of use. In particular the following plasmids have been used for efficient expression of functional nucleic acid molecules.

Mammalian Expression Plasmids:
Plasmid Name: pCDNA3.1 (−)
Expression: CMV promoter
   BGH poly(A) terminator
Plasmid Name: pDUAL-eGFPΔ (modified from peGFP-C2)
Expression: H1 promoter
   BGH poly(A) terminator

Viral Vectors:
Vector Name: pAAV
Virus: Adeno-Associated Virus
Expression: CAG promoter/CMV enhancer
   SV40 late poly(A) terminator
Vector Name: rcLV-TetOne-Puro
Virus: Lentivirus (3rd generation)
Expression: LTR-TREt (Tre-Tight) promoter (doxycycline-inducible expression)
   BGH poly(A) terminator
Vector Name: pLPCX-link
Virus: Retrovirus (3rd generation)
Expression: CMV

It should be noted that any promoter may be used in the vector and will work just as well as those mentioned above.

Compositions

The present invention also relates to compositions comprising the above said functional nucleic acid molecules or the above said DNA molecules. Any compositions are included allowing to deliver the above said functional nucleic acid molecules by viral vectors (AAV, lentivirus and the like) and non-viral vectors (nanoparticles, lipid particles and the like).

According to a further aspect of the invention, there is provided the functional nucleic acid molecule, DNA molecule, expression vector or the composition as defined herein for use as a medicament.

It will be understood that the functional nucleic acid molecules of the invention find use in increasing the level of frataxin protein within a cell. Therefore, the above said functional nucleic acid molecules, DNA molecules and/or compositions may be used as medicaments, preferably for treating Friedreich's ataxia and in particular promoting the recovery of disease-associated mitochondrial defects.

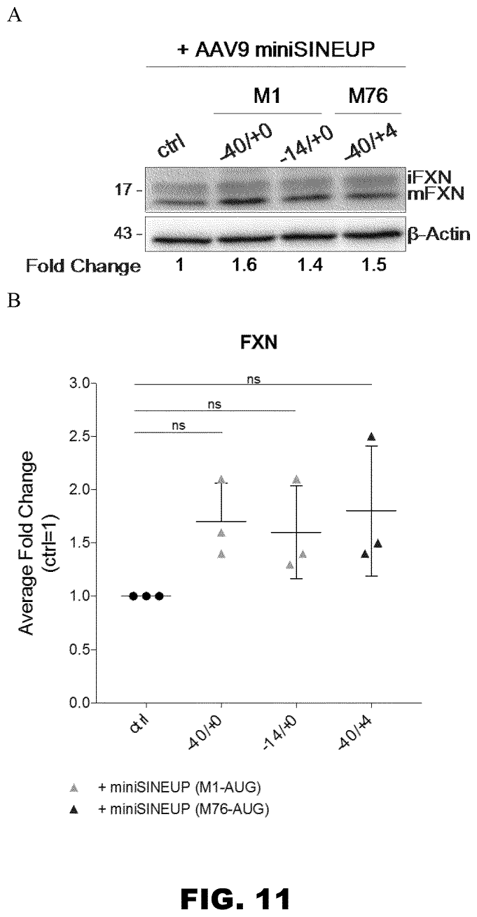

Friedreich's ataxia is a rare genetic disorder caused by an insufficient quantity of frataxin protein. The main root of the pathology is the impaired transcription of the FXN gene as a result of GAA repeat expansion. As shown in the Examples provided herein, the functional nucleic acid molecules were able to rescue the physiological translation of frataxin even in patient cells with a mRNA deficit (e.g. see FIG. 11A showing Western blot comparison between healthy, patient lymphoblasts and patient lymphoblasts stably expressing miniSINEUP-FXN). Use of the functional nucleic acid molecules to treat Friedreich's ataxia has several advantages including inducing target gene expression within the range of 1.5 to 2.5 fold thus limiting side effects due to exaggerated overexpression and enabling exclusively in situ translation enhancement avoiding ectopic protein synthesis in the absence of the target mRNA. They also do not trigger any hereditable genome editing.

According to a further aspect of the invention, there is provided the use of the functional nucleic acid molecule (or DNA molecule, expression vector or composition) as defined herein for the manufacture of a medicament for the treatment of Friedreich's ataxia.

Methods

According to a further aspect of the invention, there is provided a method for enhancing protein translation of FXN mRNA in a cell comprising administering the functional nucleic acid molecule, DNA molecule, expression vector or composition as defined herein to the cell. Preferably the cell is a mammalian cell, such as a human or a mouse cell.

According to a further aspect of the invention, there is provided a method for increasing the protein synthesis efficiency of frataxin in a cell comprising administering the functional nucleic acid molecule, DNA molecule, expression vector or composition as defined herein to the cell.

The methods described herein may comprise transfecting into a cell the functional nucleic acid molecule, DNA molecule or expression vector as defined herein. The functional nucleic acid molecule, DNA molecule or expression vector may be administered to target cells using methods known in the art and include, for example, microinjection, lipofection, electroporation, using calcium phosphate, self-infection by the vector or transduction of a virus.

The target cell to be treated may comprise a reduced amount of frataxin. In one embodiment, the level of frataxin in the cell is lower than the level of frataxin in a normal cell (i.e. a cell comprising a normal phenotype with functional copies of the FXN gene). For example, the level of frataxin in the cell may be less than 70% of the level of frataxin in a normal cell, such as less than 60% or less than 50% of the level of frataxin in a normal cell. In a further embodiment, the level of frataxin in the cell is about 50% of the level of frataxin in a normal cell.

In a further embodiment, the cell is FXN haploinsufficient, i.e. wherein the presence of a variant allele in a heterozygous combination results in the amount of product generated by the single wild-type gene is not sufficient for complete or normal function. Generally, haploinsufficiency is a condition that arises when the normal phenotype requires the protein product of both alleles, and reduction to 50% or less of gene function results in an abnormal phenotype.

Methods of the invention result in increased levels of frataxin in a cell and therefore find use, for example, in methods of treatment for diseases which are associated with FXN defects (i.e. reduced frataxin levels and/or loss-of-function mutations of the FXN gene). Methods of the invention find particular use in diseases caused by a quantitative decrease in the predetermined, normal protein level. Methods of the invention can be performed in vitro, ex vivo or in vivo.

According to a further aspect of the invention, there is provided a method of treating Friedreich's ataxia comprising administering a therapeutically effective amount of the functional nucleic acid molecule, DNA molecule, expression vector or composition as defined herein.

It will be understood that the embodiments described herein may be applied to all aspects of the invention, i.e. the embodiment described for the functional nucleic acid molecules may equally apply to the claimed methods and so forth.

The invention will now be illustrated with reference to the following non-limiting examples.

### EXAMPLES

### Example 1

This example shows how the regulatory sequences and the target binding sequences of the functional nucleic acid according to the invention have been designed.

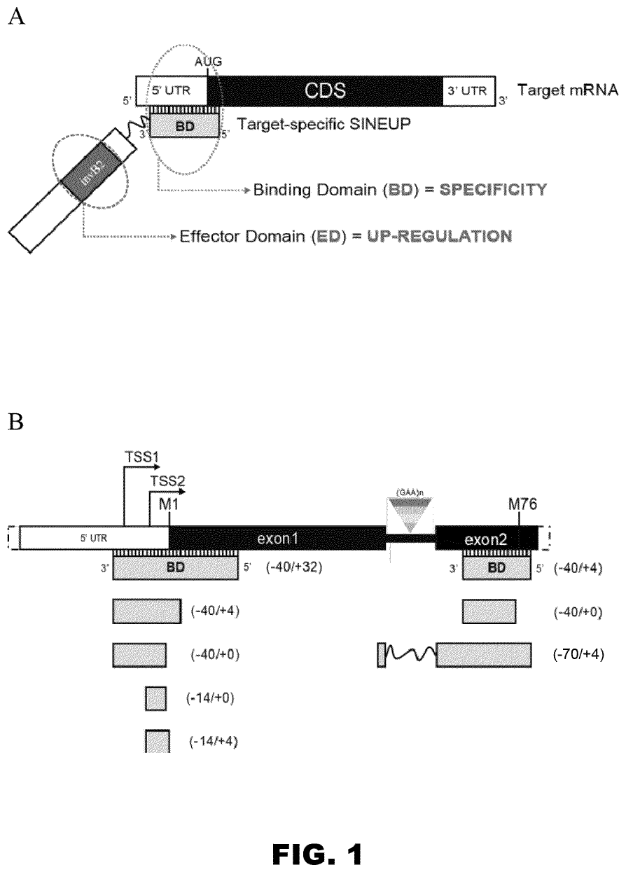

In particular, FIG. 1A shows a schematic representation of SINEUPs functional domains. The binding domain (BD, grey) provides SINEUP specificity and is in antisense orientation to the sense protein-coding mRNA (Target mRNA). The inverted SINEB2 element (invB2) is the effector domain (ED) and confers enhancement of protein synthesis. 5′ to 3′ orientation of sense and antisense RNA molecules is indicated. Structural elements of target mRNA are shown: 5′ untranslated region (5′UTR, white), coding sequence (CDS, black) and 3′ untranslated region (3′UTR, white). Scheme is not drawn in scale. FIG. 1B shows a scheme of human FXN gene (5′-end, white) and BDs (grey) design of synthetic SINEUP-FXN targeting the initiating M1-AUG and the M76-AUG downstream GAA expansions. The numbering refers to the position according to the methionine (i.e. −40/+32, from 40 nucleotides upstream and to 32 nucleotides downstream the M1-AUG). Scheme is not drawn in scale.

The sequences used in the examples are as follows.

| SEQ ID | | |
|---|---|---|
| NO. | Definition | Features |
| 83 | SINEUP-FXN in antisense | BD = −40/+32 relative to M1-AUG (72 bp) |
| | orientation to FXN mRNA | ED = inverted SINEB2 repeat (167 bp) |
| | isoform 1 | Partial Alu element (45 bp) |
| | | Extra sequence (13 bp) |
| | | Backbone = Δ5'ASUchl1 (1105 bp) |
| 84 | SINEUP-FXN in antisense | BD = −40/+4 relative to M1-AUG (44 bp) |
| | orientation to FXN mRNA | ED = inverted SINEB2 repeat (167 bp) |
| | isoform 1 | Partial Alu element (45 bp) |
| | | Extra sequence (13 bp) |
| | | Backbone = Δ5'ASUchl1 (1105 bp) |
| 85 | SINEUP-FXN in antisense | BD = −21/+32 relative to M1-AUG (53 bp) |
| | orientation to FXN mRNA | ED = inverted SINEB2 repeat (167 bp) |
| | isoform 1 | Partial Alu element (45 bp) |
| | | Extra sequence (13 bp) |
| | | Backbone = Δ5'ASUchl1 (1105 bp) |
| 86 | SINEUP-FXN in antisense | BD = −21/+4 relative to M1-AUG (25 bp) |
| | orientation to FXN mRNA | ED = inverted SINEB2 repeat (167 bp) |
| | isoform 1 | Partial Alu element (45 bp) |
| | | Extra sequence (13 bp) |
| | | Backbone = Δ5'ASUchl1 (1105 bp) |
| 87 | SINEUP-FXN in antisense | BD = −40/+0 relative to M1-AUG (40 bp) |
| | orientation to FXN mRNA | ED = inverted SINEB2 repeat (167 bp) |
| | isoform 1 | Partial Alu element (45 bp) |
| | | Extra sequence (13 bp) |
| | | Backbone = Δ5'ASUchl1 (1105 bp) |
| 88 | SINEUP-FXN in antisense | BD = −14/+0 relative to M1-AUG (14 bp) |
| | orientation to FXN mRNA | ED = inverted SINEB2 repeat (167 bp) |
| | isoform 1 | Partial Alu element (45 bp) |
| | | Extra sequence (13 bp) |
| | | Backbone = Δ5'ASUchl1 (1105 bp) |
| 89 | SINEUP-FXN in antisense | BD = −14/+4 relative to M1-AUG (18 bp) |
| | orientation to FXN mRNA | ED = inverted SINEB2 repeat (167 bp) |
| | isoform 1 | Partial Alu element (45 bp) |
| | | Extra sequence (13 bp) |
| | | Backbone = Δ5'ASUchl1 (1105 bp) |
| 90 | SINEUP-FXN in antisense | BD = −40/+4 relative to M76-AUG (44 bp) |
| | orientation to FXN mRNA | ED = inverted SINEB2 repeat (167 bp) |
| | isoform 1 | Partial Alu element (45 bp) |
| | | Extra sequence (13 bp) |
| | | Backbone = Δ5'ASUchl1 (1105 bp) |
| 91 | SINEUP-FXN in antisense | BD = −40/+4 relative to M76-AUG (40 bp) |
| | orientation to FXN mRNA | ED = inverted SINEB2 repeat (167 bp) |
| | isoform 1 | Partial Alu element (45 bp) |
| | | Extra sequence (13 bp) |
| | | Backbone = Δ5'ASUchl1 (1105 bp) |
| 92 | SINEUP-FXN in antisense | BD = (−10/−60/+ 0 relative to |
| | orientation to FXN mRNA | M76-AUG (70 bp) |
| | isoform 1 | ED = inverted SINEB2 repeat (167 bp) |
| | | Partial Alu element (45 bp) |
| | | Extra sequence (13 bp) |
| | | Backbone = Δ5'ASUchl1 (1105 bp) |
| 93 | miniSINEUP-FXN in | BD = −40/+0 relative to M1-AUG (40 bp) |
| | antisense orientation to FXN | ED = inverted SINEB2 repeat (167 bp) |
| | mRNA isoform 1 | Extra sequence (13 bp) |
| 94 | miniSINEUP-FXN in | BD = −14/+0 relative to M1-AUG (14 bp) |
| | antisense orientation to FXN | ED = inverted SINEB2 repeat (167 bp) |
| | mRNA isoform 1 | Extra sequence (13 bp) |
| 95 | miniSINEUP-FXN in | BD = −14/+4 relative to M1-AUG (18 bp) |
| | antisense orientation to FXN | ED = inverted SINEB2 repeat (167 bp) |
| | mRNA isoform 1 | Extra sequence (13 bp) |
| 96 | miniSINEUP-FXN in | BD = −40/+4 relative to M76-AUG (44 bp) |
| | antisense orientation to FXN | ED = inverted SINEB2 repeat (167 bp) |
| | mRNA isoform 1 | Extra sequence (13 bp) |
| 97 | microSINEUP-FXN in | BD = −40/+0 relative to M1-AUG (40 bp) |
| | antisense orientation to FXN | ED = nt 44-120 of inverted SINEB2 |
| | mRNA isoform 1 | element (77 bp) |
| | | Extra sequence (19 bp) |
| 98 | microSINEUP-FXN in | BD = −14/+0 relative to M1-AUG (14 bp) |
| | antisense orientation to FXN | ED = nt 44−120 of inverted SINEB2 |
| | mRNA isoform 1 | element (77 bp) |
| | | Extra sequence (19 bp) |

BD: Binding domain; ED: Effector domain

### Example 2

This example shows that synthetic SINEUPs increase endogenous frataxin protein level in human cells in vitro. In particular, HEK 293T/17 cells (ATCC Cat. No. CRL-11268) were transfected with empty vector (ctrl) and SINEUP-FXN variants and harvested 48 hours post transfection. Empty vector (ctrl) and miniSINEUP-FXN −40/+4 M1-AUG were taken as negative and positive controls respectively. HEK 293T/17 cells were used to screen the activity of SINEUP-FXN because they endogenously express frataxin.

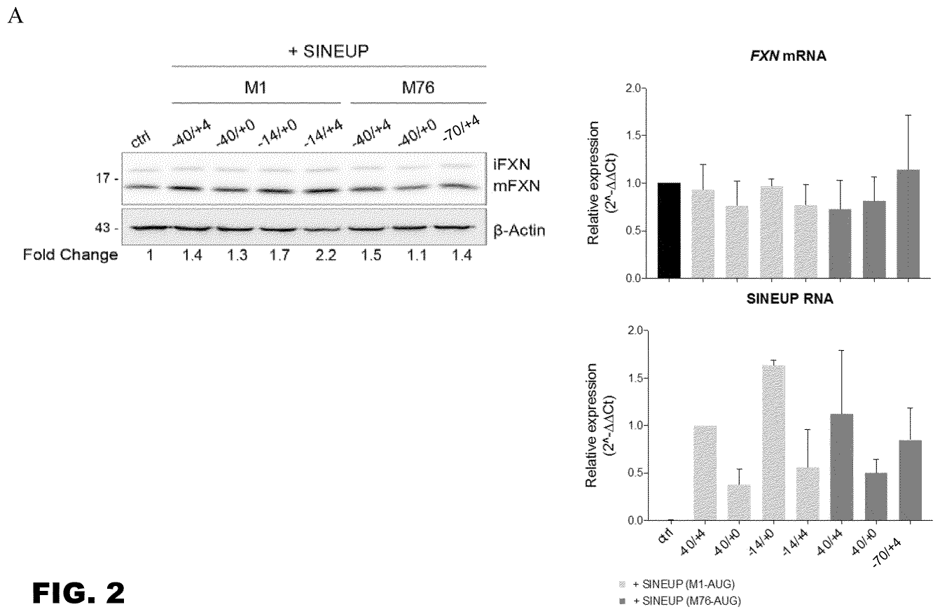

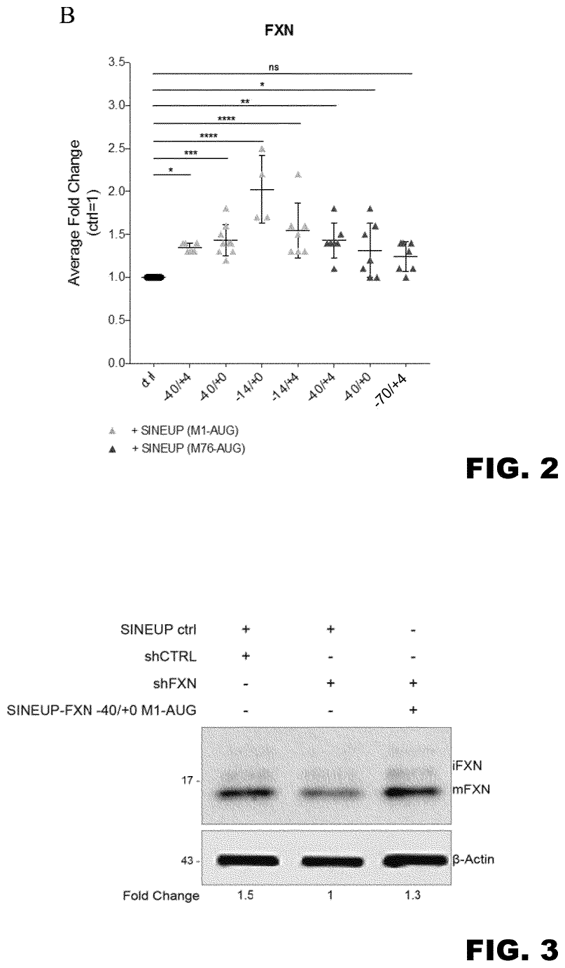

FIG. 2A, left panel shows a Western blot with anti-FXN and anti-β-actin antibodies of whole cell lysates. One representative experiment is shown. First, FXN band intensity was normalized to the relative β-actin band. Then, fold change values were calculated normalizing to control cells (ctrl). SINEUP-FXN-transfected cells show increased levels of endogenous FXN protein. FIG. 2A, right panel shows real-time PCR analysis of FXN mRNA and SINEUP RNA expression in transfected cells. In particular, BDs with minimal (−40/+4 M1-AUG) or no (−40/+0 M1-AUG) overlap to the CDS induced up-regulation of mature frataxin in the range of 1.4-fold. When the overlapping region corresponded exactly to the 5′UTR, as in the case for −14/+0 and −14/+4 M1-AUG configurations, SINEUPs reached the highest potency (1.5- to 2-fold increase). Columns represent mean±S.E.M. of n≥4 independent experiments. Variation in both target and SINEUP mRNA expression among samples are not statistically significant (one-way ANOVA followed by Dunnett's post-test). FXN transcripts were quantified, using human GAPDH (hGAPDH) expression as internal control. The FXN/hGAPDH ratio for ctrl sample was set as a baseline value to which all transcripts levels were normalized. Unchanged FXN mRNA levels are shown, thereby confirming FXN increased protein synthesis at post-transcriptional level. SINEUP transcripts were quantified, using hGAPDH expression as internal control. The SINEUP/hGAPDH ratio for −40/+4 M1-AUG sample was set as a baseline value to which all transcripts levels were normalized. FIG. 2B shows average fold change of FXN protein levels. Columns represent mean±S.E.M. of n≥4 independent experiments; ns, p>0.05; *p<0.05; **p<0.01; ***p<0.001; ****p<0.0001 (one-way ANOVA followed by Dunnett's post-test).

### Example 3

This example shows SINEUP effect on FXN knockdown HEK293T/17 cells. Silencing of FXN by shFXN (sh, Short Hairpin) in HEK 293T/17 cells. Cells were co-transfected with shCTRUSINEUP ctrl (empty vectors), shFXN/SINEUP ctrl and shFXN/SINEUP-FXN −40/+0 M1-AUG. shCTRL/SINEUP and shFXN/SINEUP were taken as negative and positive silencing controls respectively. 48 hours post transfection, whole cell lysates were analysed by Western blotting with anti-FXN and anti-β-actin antibodies (FIG. 3). First, FXN band intensity was normalized to the relative f3-actin. Then, fold change values were calculated normalizing to shCTRL/SINEUP ctrl sample. SINEUP-FXN −40/+0 M1-AUG drove a 30% increase in FXN protein level (n=1).

### Example 4

This example shows that miniSINEUPs increase endogenous frataxin protein level in human cells in vitro.

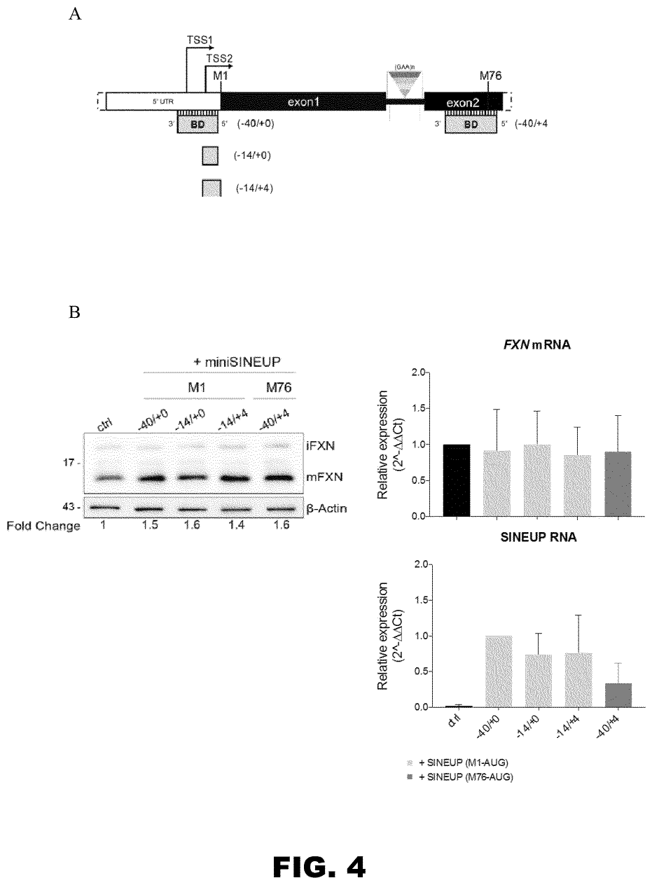

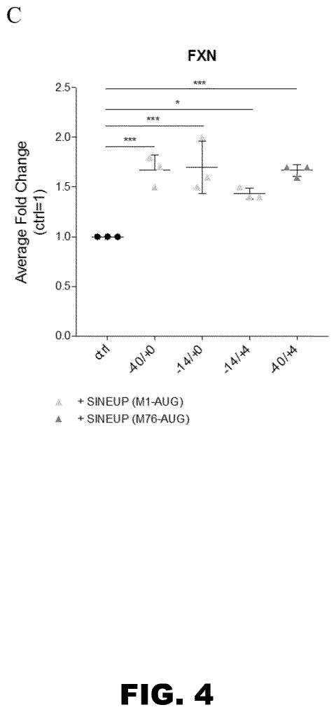

In particular FIG. 4A is a scheme of human FXN gene (5′-end) and binding domains anatomy of tested synthetic miniSINEUP-FXN targeting the initiating M1-AUG and the M76-AUG downstream GAA expansions. In FIGS. 4B and 4C HEK 293T/17 cells were transfected with empty vector (ctrl) and miniSINEUP-FXN variants (−40/+0; −14/+0 and −14/+4 M1-AUG or −40/+4 M76-AUG) and harvested 48 hours post transfection. Empty vector (ctrl) and miniSINEUP-FXN −40/+0 M1-AUG were taken as negative control and positive controls respectively. In FIG. 4B, left panel: 48 hours post transfection, whole cell lysates were analysed by Western blotting with anti-FXN and anti-β-actin antibodies. One representative experiment is shown. First, FXN band intensity was normalized to the relative β-actin band. Then, fold change values were calculated normalizing to control cells (ctrl). miniSINEUP-FXN-transfected cells show increased levels of endogenous FXN protein. miniSINEUPs-FXN promoted a protein induction consistently in the range of 1.4- to 1.7-fold, proving they retain the same efficacy of their full-length counterpart with the advantage of being shorter. In FIG. 4B, right panel: real-time PCR analysis of FXN mRNA and miniSINEUP RNA expression in transfected cells. Columns represent mean±S.E.M. of n=3 independent experiments. Variation in both target and SINEUP mRNA expression among samples are not statistically significant (one-way ANOVA followed by Dunnett's post-test). FXN transcripts were quantified, using human GAPDH (hGAPDH) expression as internal control. The FXN/hGAPDH ratio for ctrl sample was set as a baseline value to which all transcripts levels were normalized. Unchanged FXN mRNA levels are shown, thereby confirming FXN increased protein synthesis at post-transcriptional level. miniSINEUP transcripts were quantified, using hGAPDH expression as internal control. The miniSINEUP/hGAPDH ratio for −40/+0 M1-AUG sample was set as a baseline value to which all transcripts levels were normalized. FIG. 4C shows average fold change of FXN protein levels. Columns represent mean±S.E.M. of n=3 independent experiments; ns, p>0.05; *p<0.05; **p<0.01; ***p<0.001; ****p<0.0001 (one-way ANOVA followed by Dunnett's post-test).

### Example 5

This example shows that miniSINEUPs increase endogenous frataxin protein level in SH-SY5Y cells in vitro.

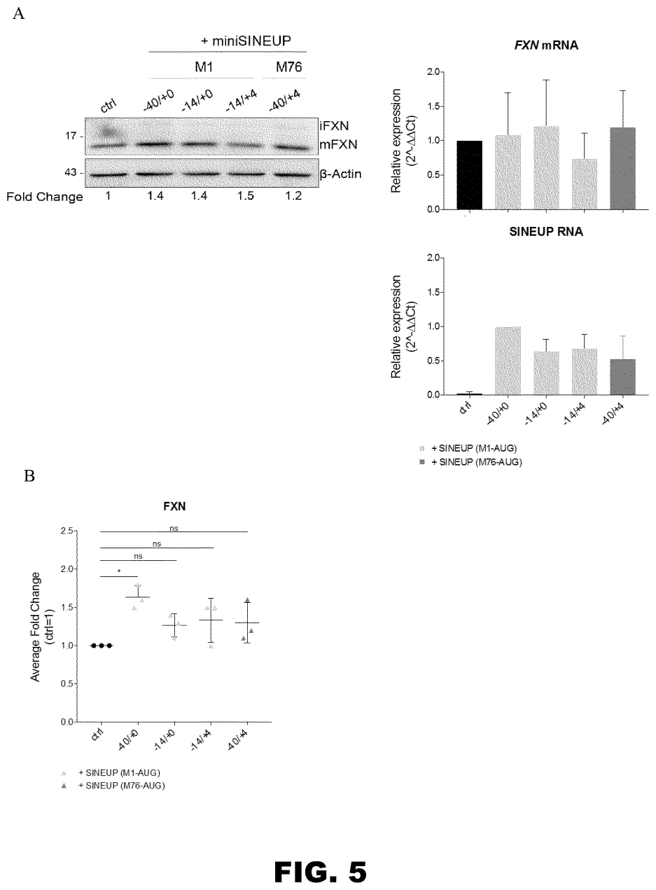

In FIGS. 5A and 5B, SH-SY5Y cells (ATCC Cat. No. CRL-2266) were transfected with empty vector (ctrl) and miniSINEUP-FXN variants (−40/+0; −14/+0 and −14/+4 M1-AUG or −40/+4 M76-AUG) and harvested 48 hours post transfection. Empty vector (ctrl) and miniSINEUP-FXN −40/+0 M1-AUG were taken as negative control and positive controls respectively. In FIG. 5A, left panel: whole cell lysates were analysed by Western blotting with anti-FXN and anti-β-actin antibodies. One representative experiment is shown. First, FXN band intensity was normalized to the relative β-actin band. Then, fold change values were calculated normalizing to control cells (ctrl). miniSINEUP-FXN-transfected cells show increased levels of endogenous FXN protein. However, the up-regulation is not statistically significant. In FIG. 5A, right panel: real-time PCR analysis of FXN mRNA and miniSINEUP RNA expression in transfected cells. Columns represent mean±S.E.M. of n=3 independent experiments. Variation in both target and miniSINEUP mRNA expression among samples are not statistically significant (one-way ANOVA followed by Dunnett's post-test). FXN transcripts were quantified, using human GAPDH (hGAPDH) expression as internal control. The FXN/hGAPDH ratio for ctrl sample was set as a baseline value to which all transcripts levels were normalized. Unchanged FXN mRNA levels are shown, thereby confirming FXN increased protein synthesis at post-transcriptional level (top). minSINEUP transcripts were quantified, using hGAPDH expression as internal control. The miniSINEUP/hGAPDH ratio for −40/+0 M1-AUG sample was set as a baseline value to which all transcripts levels were normalized (bottom). FIG. 5B shows average fold change of FXN protein levels. Columns represent mean±S.E.M. of n=3 independent experiments; ns, p>0.05; *p<0.05; **p<0.01; ***p<0.001; ****p<0.0001 (one-way ANOVA followed by Dunnett's post-test).

### Example 6

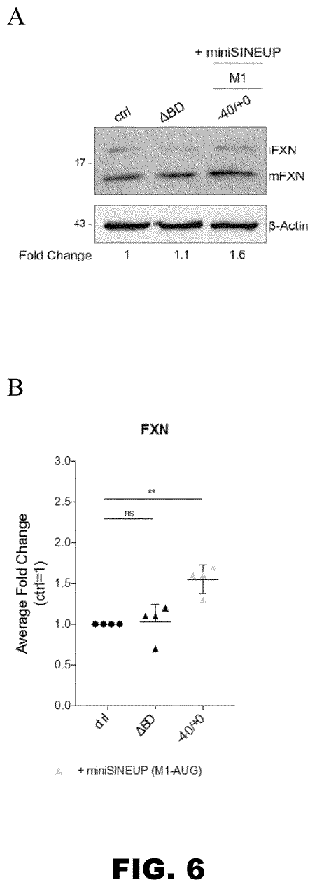

This example shows the binding domain's specificity: a selective increase of frataxin protein expression in vitro is demonstrated. In FIGS. 6A and 6B, HEK 293T/17 cells were transfected with empty vector (ctrl), deltaBD (ΔBD, construct lacking the overlapping region to FXN mRNA), miniSINEUP-FXN −40/+0 M1-AUG and harvested 48 hours post transfection. Empty vector (ctrl) and miniSINEUP-FXN −40/+0 M1-AUG were taken as negative control and positive controls respectively. In FIG. 6A, whole cell lysates were analysed by Western blotting with anti-FXN and anti-β-actin antibodies. One representative experiment is shown. First, FXN band intensity was normalized to the relative β-actin band. Then, fold change values were calculated normalizing to control cells (ctrl). miniSINEUP-FXN-transfected cells show increased levels of endogenous FXN protein, while deltaBD-transfected cells showed unchanged protein levels. FIG. 6B shows average fold change of FXN protein levels. Columns represent mean±S.E.M. of n≥4 independent experiments; ns, p>0.05; *p<0.05; **p<0.01; ***p<0.001; ****p<0.0001 (one-way ANOVA followed by Dunnett's post-test).

### Example 7

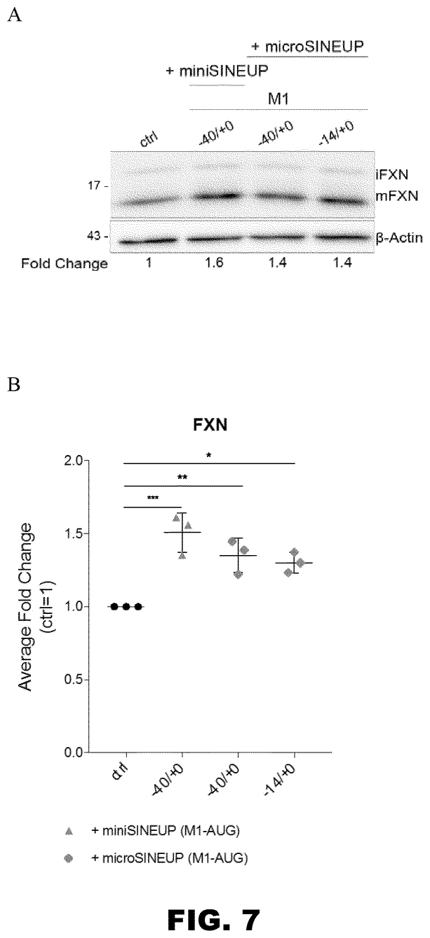

This example shows effector domain optimization. microSINEUPs increase endogenous frataxin protein level in HEK 293T/17 cells in vitro. HEK 293T/17 cells were transfected with empty vector (ctrl), miniSINEUP-FXN −40/+0 M1-AUG and microSINEUP-FXN variants (−40/+0; −14/+0 M1-AUG). Cells were harvested 48 hours post transfection. Empty vector (ctrl) and miniSINEUP-FXN −40/+0 M1-AUG were taken as negative control and positive controls respectively. In FIG. 7A, whole cell lysates were analysed by Western blotting with anti-FXN and anti-β-actin antibodies. One representative experiment is shown. First, FXN band intensity was normalized to the relative β-actin. Then, fold change values were calculated normalizing to control cells (ctrl). microSINEUP-FXN-transfected cells show increased levels of endogenous FXN protein. FIG. 7B shows average fold change of FXN protein levels. Columns represent mean±S.E.M. of n=3 independent experiments; ns, p>0.05; *p<0.05; **p<0.01; ***p<0.001; ****p<0.0001 (one-way ANOVA followed by Dunnett's post-test).

### Example 8

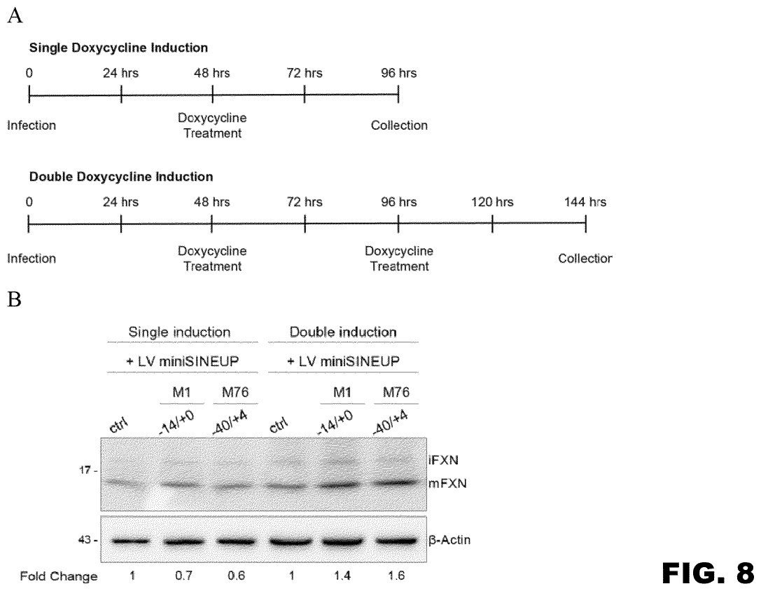

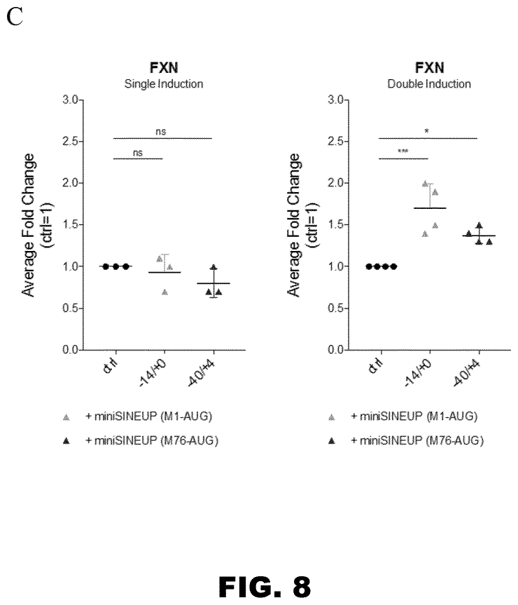

This example shows miniSINEUPs lentiviral transduction optimization. In particular, FIGS. 8A to 8C show infection of human neuroblastoma cells (SH-SY5Y) with inducible lentiviral vectors driving the expression of empty virus (ctrl) and LVminiSINEUP-FXN variants (−14/+0 M1-AUG or −40/+4 M76-AUG). FIG. 8A shows doxycycline treatment timelines. Single induction timing (top). 48 hrs after infection (time 0), cells were subjected to doxycycline treatment and harvested 96 hrs after infection. Double induction timing (bottom). Cells were treated twice with doxycycline (time 48 and 96 hrs) and harvested 144 hrs after infection. In FIG. 8B SH-SY5Y cells were infected following both protocols timing. Whole cell lysates were analysed by western blotting with anti-FXN and anti-β-actin antibodies. One representative experiment is shown. First, FXN band intensity was normalized to the relative β-actin. Then, fold change values were calculated normalizing to control cells (ctrl). LVminiSINEUP-FXN-infected cells show increased levels of endogenous FXN protein only with double doxycycline induction, while no up-regulation was observed after a single treatment. FIG. 8C shows average fold change of FXN protein levels. Columns represent mean±S.E.M. of n≥3 independent experiments; ns, p>0.05; *p<0.05; **p<0.01; ***p<0.001; ****p<0.0001 (one-way ANOVA followed by Dunnett's post-test).

### Example 9

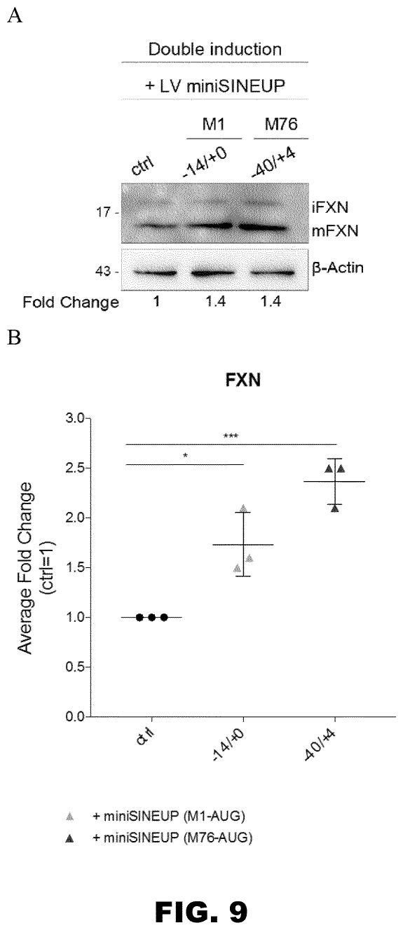

This example shows lentiviral infection of HEK293T/17 cells. In FIGS. 9A and 9B, HEK 293T/17 cells were infected with inducible lentiviral vectors driving the expression of empty virus (ctrl) and LVminiSINEUP-FXN variants (−14/+0 M1-AUG or −40/+4 M76-AUG), induced 48- and 96-hours post infection, and harvested 6 days post infection. Empty vector (ctrl) and LVminiSINEUP-FXN −40/+0 M1-AUG were taken as negative control and positive controls respectively. In FIG. 9A, whole cell lysates were analysed by western blotting with anti-FXN and anti-β-actin antibodies. One representative experiment is shown. First, FXN band intensity was normalized to the relative β-actin. Then, fold change values were calculated normalizing to control cells (ctrl). LVminiSINEUP-FXN-infected cells show increased levels of endogenous FXN protein. FIG. 9B shows average fold change of FXN protein levels. Columns represent mean±S.E.M. of n=3 independent experiments; ns, p>0.05; *p<0.05; **p<0.01; ***p<0.001; ****p<0.0001 (one-way ANOVA followed by Dunnett's post-test).

### Example 10

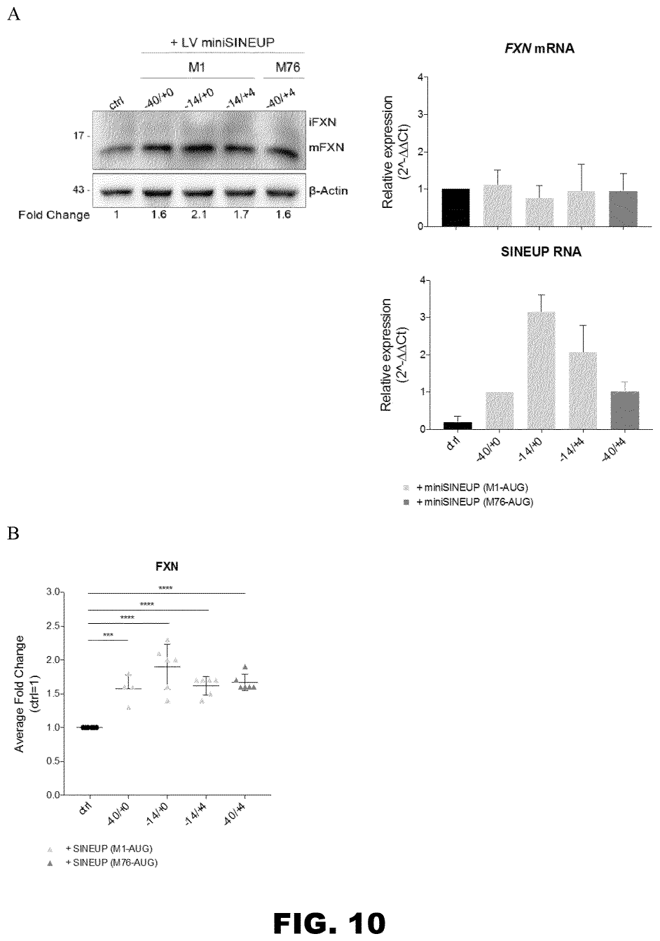

This example shows increased endogenous FXN protein expression in FRDA-derived fibroblasts. GM04078 cells (patients' primary fibroblasts) showed an intermediate phenotype carrying a hyper-expansion of about 541 repeats on one allele and 420 repeats on the other one. In FIGS. 10A and 10B GM04078 cells were infected with inducible lentiviral vectors driving the expression of empty virus (ctrl) and LVminiSINEUP-FXN variants (−40/+0; −14/+0 and −14/+4 M1-AUG or −40/+4 M76-AUG), induced 48 and 96 hours post infection, and harvested 6 days post infection. Empty vector (ctrl) and LVminiSINEUP-FXN −40/+0 M1-AUG were taken as negative control and positive controls respectively.

In FIG. 10A, left panel: whole cell lysates were analysed by Western blotting with anti-FXN and anti-β-actin antibodies. One representative experiment is shown. First, FXN band intensity was normalized to the relative β-actin. Then, fold change values were calculated normalizing to control cells (ctrl). LVminiSINEUP-FXN-infected cells show increased levels of endogenous FXN protein. All LV miniSINEUPs led to an increase in frataxin quantities in the range of 1.6- to 2.1 fold (FIG. 10A). Importantly, the position of SINEUP BD relative to the GAA expansion and the presence of the pathological expansion itself did not interfere with the observed protein increase in patients' cells. Considering that GM04078 cells show reduced levels of frataxin, averaging around 40% when compared to age- or sex-matched healthy-derived cells (Gomez-Sebastian et al. (2007) Mol. Ther., 15: 248-254), SINEUP activity rescued physiological protein quantities in this FRDA cellular model.

In FIG. 10A, right panel: real-time PCR analysis of FXN mRNA and miniSINEUP RNA expression in transfected cells. Columns represent mean±S.E.M. of n≥4 independent experiments. Variation in both target and miniSINEUP mRNA expression among samples are not statistically significant (one-way ANOVA followed by Dunnett's post-test). FXN transcripts were quantified, using human GAPDH (hGAPDH) expression as internal control. The FXN/hGAPDH ratio for ctrl sample was set as a baseline value to which all transcripts levels were normalized. Unchanged FXN mRNA levels are shown, thereby confirming FXN increased protein synthesis at post-transcriptional level (top). miniSINEUP transcripts were quantified, using hGAPDH expression as internal control. The miniSINEUP/hGAPDH ratio for −40/+0 M1-AUG sample was set as a baseline value to which all transcripts levels were normalized (bottom). FIG. 10B shows average fold change of FXN protein levels. Columns represent mean±S.E.M. of n≥4 independent experiments; ns, p>0.05; *p<0.05; **p<0.01; ***p<0.001; ****p<0.0001 (one-way ANOVA followed by Dunnett's post-test).

### Example 11

This example shows that AAV9-miniSINEUPs increase endogenous frataxin protein level in HEK 293T/17 cells in vitro. In FIGS. 11A and 11B, HEK 293T/17 cells were transfected with adeno-associated serotype 9 (AAV9) empty vector (ctrl) and AAV9miniSINEUP-FXN variants (−40/+0; −14/+0 and −14/+4 M1-AUG or −40/+4 M76-AUG) and harvested 48 hours post transfection. Empty vector (ctrl) and miniSINEUP-FXN −40/+0 M1-AUG were taken as negative control and positive controls respectively. In FIG. 11A, whole cell lysates were analysed by Western blotting with anti-FXN and anti-β-actin antibodies. MiniSINEUP-FXN-transfected cells show increased levels of endogenous FXN protein. One representative experiment is shown. First, FXN band intensity was normalized to the relative β-actin. Then, fold change values were calculated normalizing to control cells (ctrl). miniSINEUP-FXN-transfected cells show increased levels of endogenous FXN protein. However, the up-regulation is not statistically significant. FIG. 11B shows average fold change of FXN protein levels. Columns represent mean±S.E.M. of n=3 independent experiments; ns, p>0.05; *p<0.05; **p<0.01; ***p<0.001; ****p<0.0001 (one-way ANOVA followed by Dunnett's post-test).

### Example 12

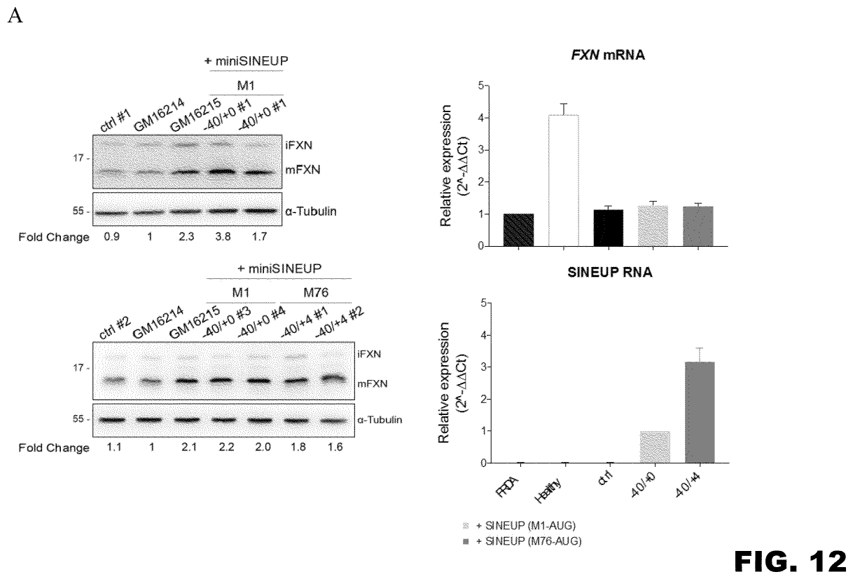

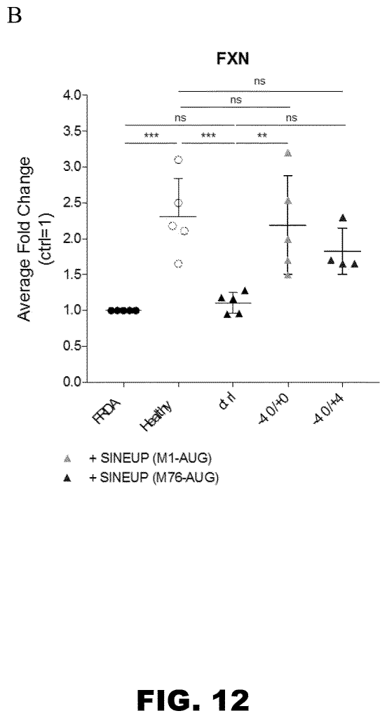

This example shows protein rescue of FRDA-derived lymphoblasts which carry the pathogenic expansion of GAA repeats and show reduced levels of the protein when compared to controls. GM16214 cells (patients' primary lymphoblasts) were stably transfected with empty vector (ctrl) and miniSINEUP-FXN variants. Ctrl, −40/+0 M1-AUG and −40/+4 M76-AUG stable clones were obtained from at least 15 days of G418 selection. Untransfected GM16214 cells and ctrl clone were taken as negative controls, while GM16215 cells (primary lymphoblasts derived from the healthy heterozygous patient's mother) were taken as positive control. In FIG. 12A, left panel: whole cell lysates were analysed by Western blotting with anti-FXN and anti-β-actin antibodies. Two representative experiments are shown. First, FXN band intensity was normalized to the relative β-actin. Then, fold change values were calculated normalizing to control cells (ctrl). GM16214 cells expressing miniSINEUP-FXN show increased levels of endogenous FXN protein. In FIG. 12A, right panel: real-time PCR analysis of FXN mRNA and miniSINEUP RNA expression in transfected cells. Columns represent mean±S.E.M. of n≥4 independent experiments. Variation in both target and miniSINEUP mRNA expression among samples are not statistically significant (one-way ANOVA followed by Dunnett's post-test). FXN transcripts were quantified, using human GAPDH (hGAPDH) expression as internal control. The FXN/hGAPDH ratio for ctrl sample was set as a baseline value to which all transcripts levels were normalized. Unchanged FXN mRNA levels are shown, thereby confirming FXN increased protein synthesis at post-transcriptional level (top). miniSINEUP transcripts were quantified, using hGAPDH expression as internal control. The miniSINEUP/hGAPDH ratio for −40/+0 M1-AUG sample was set as a baseline value to which all transcripts levels were normalized (bottom).

FIG. 12B shows average fold change of FXN protein levels. Extracts from FRDA cells showed a significant deficit of FXN protein expression averaging ˜2.3-fold when compared to control lymphoblasts derived from the healthy heterozygous patient's mother (FIG. 12B). Analysis of independent miniSINEUP clones revealed a strong rescue of frataxin levels while negative control transfectants (ctrl) showed no significative change. In particular, an up-regulation ranging from 1.6- to 2.9-fold is observed when compared to negative controls (FIG. 12B). Columns represent mean±S.E.M. of n≥4 independent experiments; ns, p>0.05; *p<0.05; **p<0.01; ***p<0.001; ****p<0.0001 (one-way ANOVA followed by Dunnett's post-test).

### Example 13

This example shows the phenotypic rescue of FRDA-derived lymphoblasts. Frataxin-deficient cells are primarily affected by defective iron-sulfur cluster biosynthesis. Accordingly, insufficient frataxin levels trigger a typical loss in the activity of aconitases, two different ISC-dependent enzymes located in mitochondrial and cytosolic compartments. To assess the functional impact of SINEUPs, aconitase activity was chosen as a functional readout of restoring frataxin physiological levels for FRDA stable transfectants. Citrate synthase assay is used as an internal control.

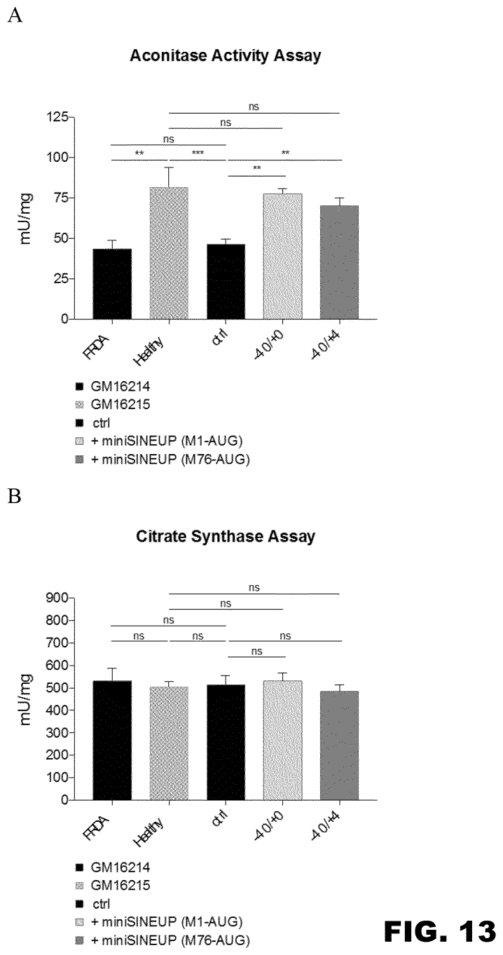

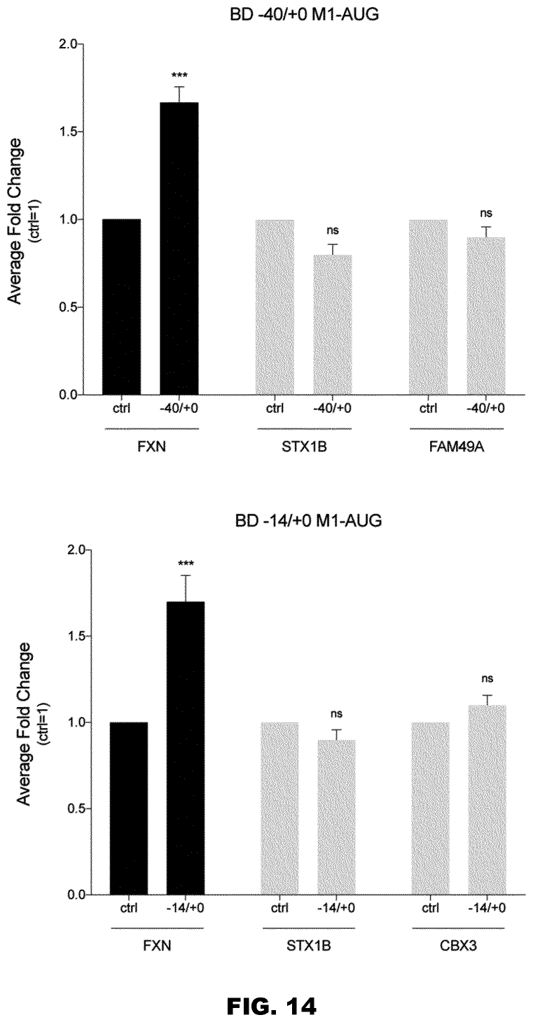

The effect of miniSINEUP-FXN expression in vitro on aconitase activity was measured on whole cells lysates. Untransfected GM16214 cells and ctrl clones were taken as negative controls, while GM16215 cells (primary lymphoblasts derived from the healthy heterozygous patient's mother) were taken as positive control, as previously shown for other experiments. GM16214 cells expressing miniSINEUP-FXN show restored activity of endogenous aconitase as compared to GM16215 positive control. Activity of citrate synthase, the Krebs cycle enzyme catalysing the preceding step respect to aconitase, but lacking ISC, did not show significant fluctuations in assayed extracts. Aconitase (FIG. 13A) and citrate synthase (FIG. 13B) activities are expressed as mU/mg ratio. Columns represent mean±S.E.M. (n≥4) of mU/mg values; ns, p>0.05; *p<0.05; **p<0.01; ***p<0.001; ****p<0.0001 (one-way ANOVA followed by Dunnett's post-test).

### Example 14

This example shows analysis of off-target effects. To predict putative off-targets, the Basic Local Alignment Search Tool (BLAST) of Ensembl genome browser was used to align the binding domain sequences to the human mRNA dataset. Results were filtered for match orientation while non-functional genes (pseudogenes and patch chromosomes) were removed. Potential off-target mRNAs were identified for their 100% identity to SINEUP-FXNs within the 5′UTRs of STX1B, FAM49A and CBX3 genes with length ranging from 13 to 20 nucleotides. All complementary sequences were distant from the translation initiation site of the target mRNA. Unconventional positions of target binding sites were also identified for TUBGCP5 (CDS) and for SH3GLB2, EIF4E and DISC1 (3′UTRs).

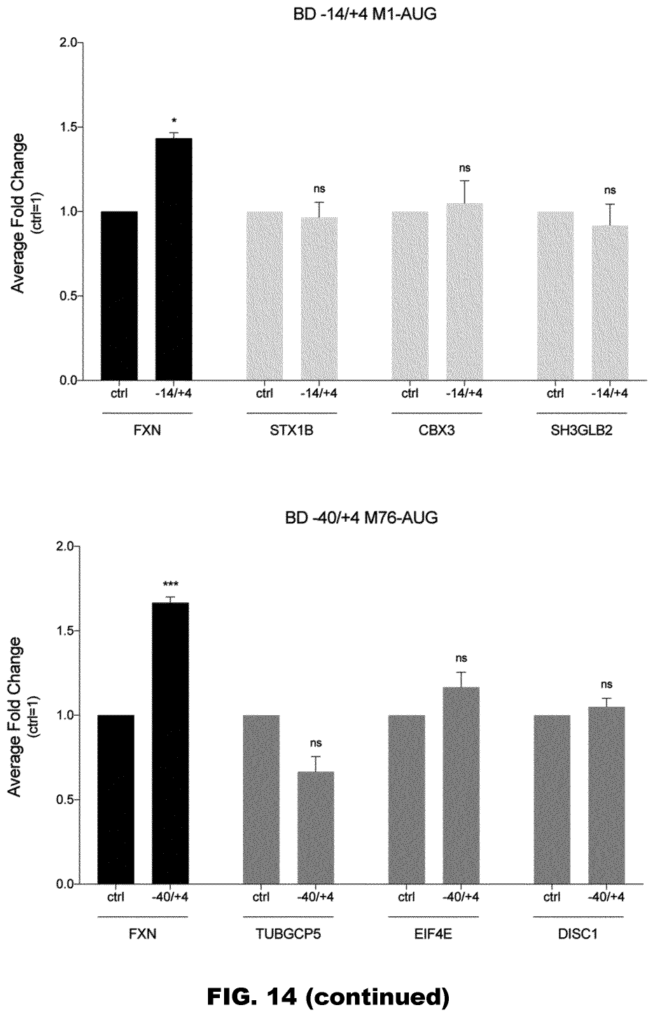

HEK 293T/17 cells were transfected with empty vector (ctrl) and miniSINEUP-FXN variants (−40/+0; −14/+0 and −14/+4 M1-AUG or −40/+4 M76-AUG) and harvested 48 hours post transfection. Empty vector (ctrl) was taken as negative control. Whole cell lysates were analysed by western blotting. Average fold changes of both target and off-targets for each binding domain are shown. First, band intensity was normalized to the relative β-actin band. Then, fold change values were calculated normalizing to control cells (ctrl). The mean FXN fold changes are plotted as the mean±S.E.M. (n≥4); ns, P>0.05; *P<0.05; **P<0.01; ***P<0.001; ****P<0.0001 (one-way ANOVA followed by Dunnett's post-test). Off-targets fold changes are plotted as the mean±SEM (n=3) and p values are calculated by unpaired t-test with Welch's correction. ns, P>0.05. As shown in FIG. 14, in the very same lysates where miniSINEUP-FXNs were active, no effects on these additional targets were observed highlighting the specificity of the designed SINEUP RNAs.

The above disclosed examples show that a number of antisense long non-coding SINEUP RNAs targeting human FXN mRNA are capable to up-regulate frataxin protein to physiological amounts acting at post-transcriptional level. FXN-specific SINEUPs promote the recovery of disease-associated mitochondrial defects in FRDA-derived cells. SINEUPs are feasibly the first gene-specific therapeutic approach to activate FXN translation in FRDA.

In detail:
1) binding domains efficient to target FXN mRNA have been identified and optimised.
2) Effector domains efficient to upregulate frataxin protein levels including those indicated as miniSINEUPs and microSINEUPs and their modifications have been identified and optimised.
3) Experimental proof has been obtained of SINEUP ability to increase frataxin protein levels within physiological range, even when endogenous frataxin levels are very low as in FRDA patients.
4) The ability of SINEUP-FRXs to function when delivered with different types of plasmid and viral vectors has been shown.
5) The ability of SINEUP-FRXs to function within different types of cells, including those of FRDA patients has been shown.
6) The ability of SINEUP-FRXs to restore mitochondrial activity within physiological range in patients' cells has been shown.

The present invention therefore provides functional nucleic acids that are able to increase endogenous frataxin protein levels within physiological range and restore mitochondrial activity in FRDA patients' cells representing a new therapeutic strategy for this untreatable disease. By restoring physiological levels of frataxin, the molecules according to the invention limit potential side effects due to exaggerated overexpression of frataxin proteins by more conventional gene therapy approaches. In addition, by taking advantage of RNA base pairing between the binding domain and FRX mRNA, the molecules according to the invention limit potential side effects present when a small molecule approach is used to treat FRDA patients.

1. A functional nucleic acid molecule comprising:
at least one target binding sequence comprising a sequence reverse complementary to a frataxin mRNA sequence; and
at least one regulatory sequence comprising an RNA comprising a SINE B2 element or a functionally active fragment of a SINE B2 element or an internal ribosome entry site (IRES) sequence or an IRES derived sequence.

2. The functional nucleic acid molecule according to claim 1, wherein the at least one regulatory sequence comprises a sequence with at least 75% sequence identity with a sequence selected from the group consisting of SEQ ID NO: 1 to SEQ ID NO: 51 and SEQ ID NO: 53 to SEQ ID NO: 82.

3. The functional nucleic acid molecule according to claim 2, wherein the at least one regulatory sequence comprises a sequence with at least 90% sequence identity with a sequence selected from the group consisting of SEQ ID NO: 1 to SEQ ID NO: 51 and SEQ ID NO: 53 to SEQ ID NO: 82.

4. The functional nucleic acid molecule according to claim 1, wherein the at least one target binding sequence is at least 10 nucleotides long and comprises, from 3′ to 5′:
a sequence reverse complementary to 0 to 50 nucleotides of the 5′ untranslated region (5′ UTR) and 0 to 40 nucleotides of the coding sequence (CDS) of the frataxin mRNA sequence; or
a sequence reverse complementary to 0 to 80 nucleotides of the region upstream of an AUG site (start codon) of the frataxin mRNA and 0 to 40 nucleotides of the CDS of the frataxin mRNA sequence downstream of said AUG site.

5. The functional nucleic acid molecule according to claim 4, wherein the at least one target binding sequence is at least 14 nucleotides long and comprises, from 3′ to 5′:
a sequence reverse complementary to 0 to 40 nucleotides of the 5′ UTR and 0 to 32 nucleotides of the CDS of the frataxin mRNA sequence; or
a sequence reverse complementary to 0 to 70 nucleotides of the region upstream of an AUG site (start codon) of the frataxin mRNA and 0 to 4 nucleotides of the CDS of the frataxin mRNA sequence downstream of said AUG site.

6. The functional nucleic acid molecule according to claim 1, further comprising at least one linker sequence between the at least one target binding sequence and the at least one regulatory sequence.

7. The functional nucleic acid molecule according to claim 1, wherein the molecule is circular.

8. A DNA molecule encoding the functional nucleic acid molecule according to claim 1.

9. An expression vector comprising the functional nucleic acid molecule according to claim 1.

10. A composition comprising the functional nucleic acid molecule according to claim 1, or comprising an expression vector that comprises the functional nucleic acid molecule.

11. A method for increasing protein synthesis efficiency of frataxin in a target cell comprising administering the functional nucleic acid molecule according to claim 1, or administering an expression vector that comprises the functional nucleic acid molecule, to the target cell.

12. The method according to claim 11, wherein the target cell exhibits a level of frataxin that is lower than the level of frataxin in a normal cell.

13-14. (canceled)

15. A method of treating Friedreich's ataxia (FRDA) comprising administering, to a patient with FRDA, a therapeutically effective amount of the functional nucleic acid molecule according to claim 1, or administering to the patient a therapeutically amount of an expression vector that comprises the functional nucleic acid molecule.

16. The method according to claim 12, wherein the cell is a mammalian cell.

17. The method according to claim 16, wherein the method is performed in vivo.
